SEQUENCE LISTING (1 sequence)

SEQ ID NO: 1 - DNA, 20 bp, Artificial sequence
  Synthetic
agagtttgatcctggctcag

(19) United States
(12) Patent Application Publication
(10) Pub. No.: US 2022/0307066 A1
(43) Pub. Date: Sep. 29, 2022

(54) AN ENZYMATIC ASSAY TO MEASURE LONG-TERM ADHERENCE TO PRE EXPOSURE PROPHYLAXIS AND ANTIRETROVIRAL THERAPY

(71) Applicants: University of Washington, Seattle, WA (US); The General Hospital Corporation, Boston, MA (US)

(72) Inventors: Ayokunle Olanrewaju, Seattle, WA (US); Paul Drain, Seattle, WA (US); Jonathan Posner, Seattle, WA (US); Derin Sevenler, Boston, MA (US); Benjamin Sullivan, Seattle, WA (US); Andrew Bender, Seattle, WA (US); Jane Zhang, Seattle, WA (US); Rebecca Sandlin, Boston, MA (US)

(73) Assignees: University of Washington, Seattle, WA (US); The General Hospital Corporation, Boston, MA (US)

(21) Appl. No.: 17/618,323

(22) PCT Filed: Jun. 12, 2020
(86) PCT No.: PCT/US2020/037609

§ 371 Date: Dec. 10, 2021

Related U.S. Application Data
(60) Provisional application No. 62/861,542, filed on Jun. 14, 2019.

(51) Int. Cl. C12Q 1/48; G01N 21/64; G01N 33/53; G01N 33/58

(52) U.S. Cl. CPC C12Q 1/48; G01N 21/64; G01N 33/53; G01N 33/58; G01N 2333/91255; G01N 2333/9126

(57) ABSTRACT

The disclosure addresses methods, compositions, and kits used to detect or quantify polymerase inhibitors in biological samples. The polymerase inhibitors can be therapeutic agents, or metabolites thereof, that have been administered to a subject as part of, for example, antiretroviral therapy (ART) or pre-exposure prophylaxis (PrEP) to address potential infections by, e.g., retroviruses such as HIV and other viruses reliant on reverse transcription. These methods, compositions, and kits can be applied to monitor a subject's compliance with the indicated therapies and can inform potential adjustments to the therapies.

### CROSS-REFERENCE(S) TO RELATED APPLICATION(S)

This application claims the benefit of Provisional Application No. 62/861,542, filed Jun. 14, 2019, the disclosure of which is incorporated herein by reference in its entirety.

### STATEMENT OF GOVERNMENT LICENSE RIGHTS

This invention was made with government support under Grant Nos. AI127200 and AI136648, awarded by the National Institutes of Health. The government has certain rights in the invention.

### STATEMENT REGARDING SEQUENCE LISTING

The sequence listing associated with this application is provided in text format in lieu of a paper copy and is hereby incorporated by reference into the specification. The name of the text file containing the sequence listing is 71602_Sequence_Final_2020-06-10.txt. The text file is 1 KB; was created on Jun. 10, 2020; and is being submitted via EFS-Web with the filing of the specification.

### BACKGROUND

For nearly 40 million people living with HIV (PLHIV) and millions more at risk of acquiring HIV, antiretroviral therapy (ART) and pre-exposure prophylaxis (PrEP) can extend the length and quality of life and prevent HIV infection. As access to ART and PrEP improves globally, medication adherence increasingly becomes a challenge in HIV treatment and prevention. Poor ART adherence leads to viral rebound, emergence of drug-resistance, and treatment failure. Poor PrEP adherence reduces individual and community-level HIV prevention benefits. Roughly 30% of PLHIV receiving ART do not maintain sufficient adherence, and non-adherence rates were higher in several PrEP trials. Poor adherence occurs for several reasons including: barriers to care or medication, medication side effects, psychological problems, and poor provider-patient relationships. Clinicians, patients, and patient advocates need tools to accurately measure antiretroviral drug levels and assess interventions to improve health outcomes.

There are several approaches for measuring ART and PrEP adherence. Subjective measures of adherence, such as self-reports and surveys, pill counts and tracking of pharmacy refills, and wireless pill containers, do not provide proof of pill ingestion limiting their accuracy. Digital pills with radio frequency transmitters embedded in gel caps provide proof of pill ingestion and information about short and long-term adherence patterns. Digital pills require an individual to wear an RFID receiver that transmits the signal to a cloud-based server, and require modification of the medication which may trigger additional regulatory review and may be cost prohibitive in global health settings.

Quantifying concentrations of antiretroviral drugs and their metabolites is an objective approach to measure ART and PrEP adherence. Tenofovir disoproxil fumarate (TDF) is used in all PrEP regimens currently recommended by health organizations (e.g. WHO and US Centers for Disease Control) and tenofovir-based treatment regimens are used in over 90% of all ART regimens. TDF is hydrolyzed into tenofovir (TFV) and phosphorylated intracellularly by nucleotide kinases into tenofovir diphosphate (TFV-DP). TFV-DP is a nucleotide reverse transcriptase inhibitor (NRTI) that terminates the DNA chain when HIV reverse transcriptase (HIV RT) synthesizes complementary DNA (cDNA). TFV has a short half-life (15 hours) in plasma and is detectable for up to 7 days. TFV measurement is susceptible to the “white coat” effect, where one is unable to correctly identify patients who take their medications just before a doctor's office visit. Conversely, TFV-DP has a longer half-life (17 days) and accumulates 25-fold in red blood cells (RBCs) and thus provides adherence information over one to two months. TFV-DP drug levels are associated with health outcomes such as viral suppression and PrEP efficacy.

Immunoassays were recently developed to measure TFV. Competitive immunoassays accurately classified recent dosage (≤24 hours) and identified non-adherence that was sustained for more than 7 days. However, all the HIV adherence monitoring immunoassays developed so far have targeted TFV and as such are susceptible to the white coat effect.

TFV-DP drug levels can be measured accurately by liquid chromatography/mass spectrometry (LC/MS). Median TFV-DP concentrations ranged from 15-170 fmol/10⁶ RBCs depending on adherence. Pharmacokinetic studies with LC/MS demonstrated that PrEP clients taking ≥4 doses/week are considered to maintain long-term adherence and are protected from HIV infection. Nevertheless, LC/MS requires significant capital investment, extensive sample preparation, trained personnel, and cold reagent storage, and is unsuitable for routine clinical use.

Accordingly, despite the advances in the art of detection anti-viral therapeutic agents, there remains a need for monitoring the levels of anti-viral agents in the body of subjects, e.g., receiving ART or PrEP, to ascertain adherence to the therapeutic regimen and to adjust or optimize the therapy accordingly to maximize success. The present disclosure addresses this and related needs.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, the disclosure provides a method of detecting a polymerase inhibitor in a biological sample, the method comprises: contacting the biological sample with a single stranded nucleic acid template, a single stranded nucleic acid primer molecule that hybridizes to the nucleic acid template, a polymerase, dNTPs, and a fluorescent dye molecule. The method further comprises providing conditions sufficient to permit the polymerase to produce a double stranded nucleic acid molecule by extending a complementary strand along the nucleic acid template, and measuring fluorescence in the biological sample. A reduced level of measured fluorescence compared to a reference standard indicates the presence of a polymerase inhibitor in the biological sample.

In some embodiments, the polymerase inhibitor is a pharmaceutical agent, or metabolite or derivative thereof, and the biological sample is obtained from a subject. In some embodiments, the polymerase inhibitor is a reverse transcriptase inhibitor or a metabolite thereof. In some embodiments, the reverse transcriptase inhibitor is a nucleotide reverse transcriptase inhibitor or a metabolite thereof. In some embodiments, the reverse transcriptase inhibitor is a nucleoside reverse transcriptase inhibitor. In some embodiments, the metabolite of the reverse transcriptase inhibitor is tenofovir diphosphate (TFV-DP), azidothymidine triphosphate (AZT-TP), emtricitabine triphosphate (FTC-TP), lamivudine triphosphate (3TC-TP), adefovir diphosphate, or entecavir triphosphate.

In some embodiments, the method further comprises determining a relative concentration of polymerase inhibitor in the biological sample, wherein intensity of fluorescence is inversely correlated to the concentration of polymerase inhibitor in the biological sample.

In some embodiments, the method comprises assessing the subject's adherence to pre-exposure prophylaxis (PrEP), wherein an indicated presence of the pharmaceutical agent, or metabolite or derivative thereof, in the biological sample above a pre-set threshold indicates the subject's adherence to PrEP, or wherein a lack of indicated presence of the pharmaceutical agent, or metabolite or derivative thereof, in the biological sample above the pre-set threshold indicates the subject's non-adherence to PrEP. In other embodiments, the method comprises assessing the subject's adherence to antiretroviral therapy (ART), wherein an indicated presence of the pharmaceutical agent, or metabolite or derivative thereof, in the biological sample above a pre-set threshold indicates the subject's adherence to ART, or wherein a lack of indicated presence of the pharmaceutical agent, or metabolite or derivative thereof, in the biological sample above the pre-set threshold indicates the subject's non-adherence to ART. In other embodiments, the method comprises assessing the subject's adherence to anti-Hepatitis virus therapy, wherein an indicated presence of the pharmaceutical agent, or metabolite or derivative thereof, in the biological sample above a pre-set threshold indicates the subject's adherence to anti-Hepatitis virus therapy, or wherein a lack of indicated presence of the pharmaceutical agent, or metabolite or derivative thereof, in the biological sample above the pre-set threshold indicates the subject's non-adherence to anti-Hepatitis virus therapy.

In some embodiments, the single stranded nucleic acid template is DNA. In some embodiments, the single stranded nucleic acid template is RNA. In some embodiments, the polymerase enzyme has RNA-dependent DNA polymerase properties. In some embodiments, the polymerase enzyme has DNA-dependent DNA polymerase properties. In some embodiments, the polymerase enzyme has RNA-dependent RNA polymerase properties. In some embodiments, the single stranded DNA template comprises a primer binding domain and a chain terminating domain. In some embodiments, the polymerase inhibitor is or comprises a dATP analog and wherein the chain terminating domain comprises at least 20% thymine residues. In some embodiments, the chain terminating domain comprises between about 25% to about 70% thymine residues. In some embodiments, the polymerase inhibitor is or comprises a dCTP analog and wherein the chain terminating domain comprises at least 20% guanine residues. In some embodiments, the chain terminating domain comprises between about 25% to about 70% cytosine residues. In some embodiments, the single stranded DNA template has at least 50 nucleotides. In some embodiments, the single stranded DNA template has about 50 nucleotides to about 2000 nucleotides.

In some embodiments, the biological sample is blood, serum, plasma, urine, or saliva. In some embodiments, the biological sample comprises red blood cells and/or peripheral blood mononuclear cells (PBMCs). In some embodiments, the biological sample is blood and the method further comprises diluting the blood to a final concentration of about 0.1% to about 20%. In some embodiments, the biological sample is blood and the method further comprises heating the biological sample to above about 70° C.

In some embodiments, the dNTPs have a final concentration of at least about 20 nM. In some embodiments, the dNTPs have a final concentration of about 25 nM to about 1000 nM. In some embodiments, the fluorescent dye molecule is an intercalating dye molecule. In some embodiments, the fluorescent intercalating dye molecule is PicoGreen, ethidium bromide, SYBR Green, propidium iodide, 7-aminoactinomycin D, EvaGreen, and the like. In some embodiments, the fluorescent dye molecule is linked to a nucleic acid probe.

In some embodiments, the method further comprises obtaining the biological sample from the subject.

In another aspect, the disclosure provides a method of assessing the presence of an anti-viral therapeutic agent in a subject receiving pre exposure prophylaxis (PrEP) or antiretroviral therapy (ART) against a viral infection. The method comprises: contacting a biological sample obtained from the subject with a single stranded nucleic acid template, a single stranded nucleic acid primer molecule that hybridizes to the nucleic acid template, a reverse transcriptase (RT) enzyme, dNTPs, and a fluorescent dye molecule. The method also comprises providing conditions sufficient to permit the RT enzyme to produce a double stranded nucleic acid molecule by extending a complementary strand along the nucleic acid template, and measuring the fluorescence in the biological sample. A reduced level of measured fluorescence compared to a reference standard indicates the presence of an anti-viral therapeutic agent in the biological sample.

In some embodiments, the anti-viral therapeutic agent is a nucleotide reverse transcriptase inhibitor. In some embodiments, the anti-viral therapeutic agent is a nucleotide reverse transcriptase inhibitor agent or metabolite thereof and is selected from tenofovir diphosphate (TFV-DP) and adefovir diphosphate. In some embodiments, the anti-viral therapeutic agent is a nucleoside reverse transcriptase inhibitor or metabolite thereof and is selected from azidothymidine triphosphate (AZT-TP), lamividuine triphosphate (3TC-TP), and emtricitabine triphosphate (FTC-TP).

In some embodiments, the method comprises determining a relative concentration of the active therapeutic agent in the biological sample, wherein intensity of fluorescence is inversely correlated to the concentration of active therapeutic agent in the biological sample. In some embodiments, the method comprises assessing the subject's adherence to pre-exposure prophylaxis (PrEP), wherein an indicated presence of the anti-viral therapeutic agent in the biological sample above a pre-set threshold indicates the subject's adherence to PrEP, and wherein a lack of indicated presence of the anti-viral therapeutic agent in the biological sample above the pre-set threshold indicates the subject's non-adherence to PrEP. In some embodiments, the method further comprises determining a level the anti-viral therapeutic agent present in the biological sample. In some embodiments, the method comprises assessing the subject's adherence to pre-exposure prophylaxis (PrEP), wherein a level of anti-viral therapeutic agent in the biological sample above a pre-set threshold indicates the subject's adherence to PrEP, and wherein the level the anti-viral therapeutic agent in the biological sample below the pre-set threshold indicates the subject's non-adherence to PrEP. In some embodiments, the method comprises assessing the subject's adherence to antiretroviral therapy (ART), wherein a level of the anti-viral therapeutic agent in the biological sample above a pre-set threshold indicates the subject's adherence to ART, and wherein a level of the anti-viral therapeutic agent in the biological sample below the pre-set threshold indicates the subject's non-adherence to ART. In some embodiments, the subject has a retroviral or hepatitis viral infection.

In some embodiments, the single stranded nucleic acid template is DNA. In some embodiments, the single stranded nucleic acid template is RNA. In some embodiments, the RT enzyme has RNA-dependent DNA polymerase properties. In some embodiments, the polymerase enzyme has DNA-dependent DNA polymerase properties. In some embodiments, the polymerase enzyme has RNA-dependent RNA polymerase properties. In some embodiments, the single stranded DNA template comprises a primer binding domain and a chain terminating domain. In some embodiments, the anti-viral therapeutic agent is or comprises a dATP analog and wherein the chain terminating domain comprises at least 20% thymine residues. In some embodiments, the chain terminating domain comprises between about 25% to about 70% thymine residues. In some embodiments, the anti-viral therapeutic agent is or comprises a dCTP analog and wherein the chain terminating domain comprises at least 20% guanine residues. In some embodiments, the chain terminating domain comprises between about 25% to about 70% guanine residues. In some embodiments, the single stranded DNA template has at least 50 nucleotides. In some embodiments, the single stranded DNA template has at about 50 nucleotides to about 2000 nucleotides.

In some embodiments, the biological sample is blood, serum, plasma, urine, or saliva. In some embodiments, the biological sample comprises red blood cells and/or peripheral blood mononuclear cells (PBMCs). In some embodiments, the biological sample is blood and the method further comprises diluting the blood to a final concentration of about 0.1% to about 20%. In some embodiments, the biological sample is blood and the method further comprises heating the biological sample to above about 70° C.

In some embodiments, the dNTPs have a final concentration of at least about 20 nM. In some embodiments, the dNTPs have a final concentration of about 25 nM to about 1000 nM.

In some embodiments, the fluorescent dye molecule is an intercalating dye molecule. In some embodiments, the fluorescent intercalating dye molecule is PicoGreen, ethidium bromide, SYBR Green, propidium iodide, 7-aminoactinomycin D, EvaGreen and the like. In some embodiments, the fluorescent dye molecule is linked to a nucleic acid probe.

In some embodiments, the method further comprises obtaining the biological sample from the subject.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIGS. 1A and 1B provide a schematic overview of an exemplary embodiment of the RESTRICT Assay disclosed herein. (FIG. 1A) The RESTRICT assay requires nucleic-acid templates, primers, nucleotides, nucleotide reverse transcriptase inhibitors (NRTIs), HIV-1 reverse transcriptase enzyme (HIV RT), and intercalating dye. (FIG. 1B) The assay measures complementary DNA (cDNA) synthesis by HIV RT. At low NRTI concentrations, RT forms full-length double-stranded DNA (dsDNA) products that provide high fluorescence with intercalating dye. At intermediate NRTI concentrations, RT forms dsDNA fragments that provide intermediate fluorescence, while at high NRTI concentrations, very little (if any) dsDNA is formed resulting in low fluorescence.

FIGS. 2A-2C graphically illustrate characterization of RT activity. FIG. 2A illustrates the fluorescence intensity over time at different RT concentrations. Lines are exponential fits. N=3. FIG. 2B illustrates the effect of RT concentration on fluorescence intensity at 30 min incubation time. Fluorescence intensity plateaus above 100 nM RT. The line is a four-parameter logistic regression fit. N=3. FIG. 2C illustrates the effect of template concentration on fluorescence intensity at 100 nM RT and 30 min incubation time. The line is a linear fit of the data. N=4.

FIGS. 3A-3C graphically illustrate the RESTRICT assay in buffer. FIG. 3A illustrates RESTRICT assays with TFV-DP at different dNTP concentrations. Fluorescence intensity increases and curve shift towards larger TFV-DP concentration with dNTP concentration. FIG. 3B illustrates normalized data showing that inhibition curves shift right towards higher TFV-DP as dNTP concentration increases. Grey shaded region indicates clinical range for PrEP adherence. FIG. 3C is a graph of dNTP concentration versus IC₅₀ values. N=3, error bars indicate 95% confidence intervals.

FIGS. 4A and 4B graphically illustrate the determination of optimal blood dilution for RESTRICT assay. FIG. 4A illustrates the RT activity assay with 500 nM dNTP and diluted whole blood spiked into the assay at various final concentrations to determine how much dilution was required to minimize non-specific RT inhibition by blood matrix components. FIG. 4B illustrates the RT activity assay with 0.25% final blood concentration at low dNTP concentrations to determine the lowest dNTP concentration at which RT activity was detectable in blood.

FIGS. 5A and 5B illustrate performance of the RESTRICT assay in diluted whole blood. FIG. 5A is a flowchart for RESTRICT assay in blood. FIG. 5B is an inhibition curve with TFV-DP spiked in diluted whole blood (0.25% final concentration) and 100 nM dNTP. Grey shaded region and inset show clinical range for TFV-DP adherence. N=4, error bars indicate one standard deviation.

FIG. 6 graphically illustrates improved sample preparation results. Incorporating heat denaturation enabled semi-quantitative differentiation of clinically relevant TFV-DP levels with similar coefficient of variation in spiked blood and buffer. N=3. Error bars indicate 95% confidence intervals.

FIG. 7A and 7B graphically illustrate a comparison between RESTRICT assay and LC-MS/MS measurements. FIG. 7A illustrates that the RESTRICT assay identified participants with TFV-DP concentrations ≥700 fmol/punch. Error bars indicate 95% confidence intervals around the median. FIG. 7B illustrates that the RESTRICT assay fluorescence intensities were correlated with LC-MS/MS TFV-DP concentrations.

### DETAILED DESCRIPTION

This disclosure is based on the development of alternative strategies to detect and monitor adherence of subjects to anti-viral therapies. As described in more detail below, the inventors developed an assay, referred to a REverSe TRanscrIptase Chain Termination (RESTRICT) assay, as a rapid and accessible measurement of drug levels indicative of long term adherence to pre-exposure prophylaxis (PrEP) and antiretroviral therapy (ART). The initial embodiment of the assay incorporated designer single stranded DNA templates and intercalating fluorescent dyes to measure complementary DNA (cDNA) formation by reverse transcriptase in the presence of nucleotide reverse transcriptase inhibitor drugs. The RESTRICT assay was optimized using aqueous solutions of tenofovir diphosphate (TFV-DP), a metabolite that indicates long-term adherence to ART and PrEP, at concentrations over two orders of magnitude above and below the clinically relevant range. Dilution in water was used as a simple sample preparation strategy to detect TFV DP spiked into whole blood and accurately distinguished TFV-DP drug levels corresponding to low and high PrEP adherence. The RESTRICT assay was shown to be a fast and accessible test useful for patients and clinicians to measure and improve ART and PrEP adherence.

In accordance with the foregoing, in one aspect the disclosure provides a method of detecting a polymerase inhibitor in a biological sample. The method comprises:
contacting the biological sample with a single stranded nucleic acid template, a single stranded nucleic acid primer molecule that hybridizes to the nucleic acid template, a polymerase, dNTPs, and a fluorescent dye molecule;
providing conditions sufficient to permit the polymerase to produce a double stranded nucleic acid molecule by extending a complementary strand along the nucleic acid template; and
measuring fluorescence in the biological sample.
   A reduced level of measured fluorescence compared to a reference standard indicates the presence of a polymerase inhibitor in the biological sample.

As many antiviral therapeutics or their metabolites have properties that inhibit polymerases, the observed properties of such inhibition can be correlated and associated with the presence and/or amount of the therapeutic or metabolites thereof in the sample. This, in turn, can be associated with the status of a subject's antiviral therapeutic regimen, e.g., adherence or efficacy of pre-exposure prophylaxis (PrEP) and antiretroviral therapy (ART). Accordingly, in some embodiments the polymerase inhibitor can be a pharmaceutical agent, or metabolite or derivative thereof, wherein the biological sample is obtained from a subject that has been administered the pharmaceutical agent (e.g., in PrEP or ART regimens). In some embodiments, the method comprises actively obtaining the biological sample from the subject. The subject can be any animal being assessed for treatment and/or being treated. The subject can be a human, but can also be another mammal, particularly mammals useful as laboratory models for human disease, e.g., mouse, rat, dog, non-human primate, etc., or animals requiring veterinary treatment or prophylaxis.

As used herein, the term “polymerase inhibitor” encompasses any molecule or agent that prevents or reduces the activity of a polymerase enzyme to synthesize nucleic acid polymers compared to reaction conditions (in vivo or in vitro) without the polymerase inhibitor. The subject polymerase can have RNA-dependent DNA polymerase properties, DNA-dependent DNA polymerase properties, and/or RNA-dependent RNA polymerase properties with reference to polymerizing nucleic acids (e.g., RNA or DNA) based on an RNA or DNA template strand. In some embodiments the subject polymerase is a reverse transcriptase, which polymerizes a DNA (i.e., cDNA) strand from an RNA template strand. In such embodiments, the polymerase inhibitor can inhibit a reverse transcriptase enzyme. For example, illustrative reverse transcriptase inhibitors encompassed by the disclosure include various drugs or metabolites thereof. Such drugs include entecavir, lamivudine, adefovir, tenofovir disproxil fumarate, tenofovir alafenamide, abacavir, emtricitabine, zalcitabine, telbivudine, and the like. Exemplary, non-limiting metabolites of nucleoside reverse transcriptase inhibitor drugs encompassed by the disclosure include azidothymidine triphosphate (AZT-TP), emtricitabine triphosphate (FTC-TP), lamivudine triphosphate (3TC-TP), and entecavir triphosphate. Exemplary, non-limiting metabolites of nucleotide reverse transcriptase inhibitor drugs encompassed by the disclosure include tenofovir diphosphate (TFV-DP) and adefovir diphosphate.

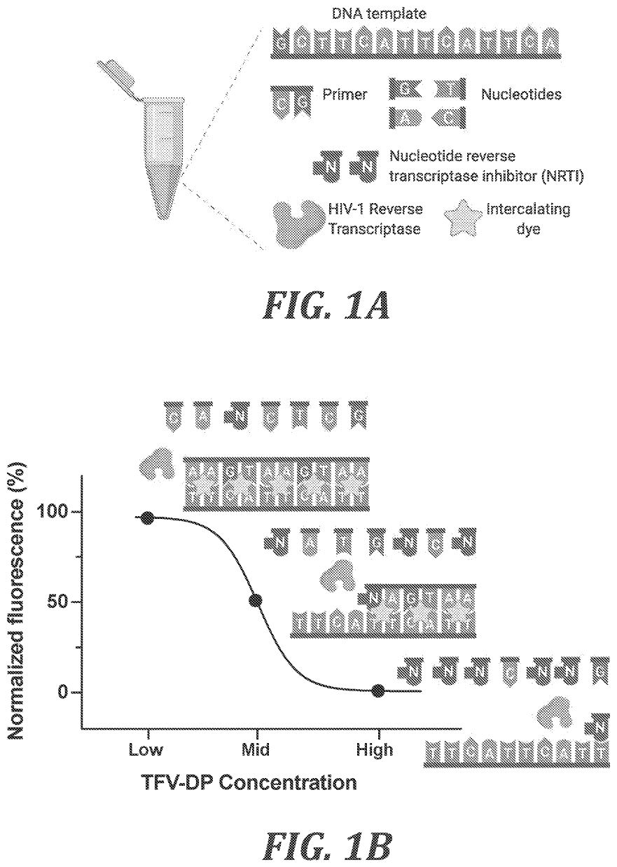

As illustrated in FIG. 1B, and described in more detail below, there is an inverse-type relationship between the presence of polymerase inhibitor and the level of fluorescence induced by polymerase activity. With higher concentration of inhibitor, there will be more extensive inhibition resulting in reduction of the polymerase activity and, therefore, reduction in the level of observed fluorescence. This trend follows the illustrated curve from the top diagram to the lower diagram (illustrated double stranded constructs not to scale). The reference standard can be or incorporate a pre-established level of fluorescence that reflects a threshold set of conditions selected by the user. In some cases, the reference standard reflects the presence of a minimal concentration of inhibitor to be effective in an anti-viral therapy, such as PrEP or ART.

In some embodiments, the method further comprises determining a relative concentration of polymerase inhibitor in the biological sample. The relative concentration can be inferred from the polymerase activity in the sample based on the extent of nucleic acid synthesis and fluorescent dye incorporation with high fluorescence indicating low inhibitor concentrations and vice-versa. Inference of the relative concentration can incorporate consideration of various factors such as the sequence of the single stranded nucleic acid template, reaction conditions (e.g., reagent concentrations, temperature), the nature of the biological sample, the processing of the biological sample, the nature of the fluorescent dye, and the like. Exemplary models that integrate such factors are described in more detail below (see, e.g., Example 2). In some embodiments, the intensity of fluorescence is inversely correlated to the concentration in polymerase inhibitor in the biological sample.

In embodiments where the biological sample is obtained from a subject and the polymerase inhibitor is a pharmaceutical agent, or metabolite or derivative thereof, the method can further comprise assessing the subject's adherence to pre-exposure prophylaxis (PrEP). PrEP refers to a prophylactic treatment where a subject is considered at risk of being exposed to an infectious agent (e.g., a virus such as hepatitis or HIV), and is administered therapeutic agents to prevent establishment if the potential exposure occurs. In such situations, the PrEP typically involves regular and routine administrations of the therapeutic agents to maintain sufficient levels of the therapeutic agent to counter any exposure to the infectious agent and prevent or minimize likelihood of an infection. If the therapeutic agents or their active metabolites are reduced due to, e.g., interruptions in the administration schedule or dose, the levels may become insufficient to prevent infection and the prophylaxis will have failed. Accordingly, in some embodiments, an indicated presence of the pharmaceutical agent, or metabolite or derivative thereof, in the biological sample above a pre-set threshold indicates the subject's adherence to PrEP. Alternatively, a lack of indicated presence of the pharmaceutical agent, or metabolite or derivative thereof, in the biological sample above the pre-set threshold indicates the subject's non-adherence to PrEP. The pre-set threshold can be established by persons of ordinary skill in the art to reflect amounts of therapeutic agent known to be effective in PrEP.

In other embodiments where the biological sample is obtained from a subject and the polymerase inhibitor is a pharmaceutical agent, or metabolite or derivative thereof, the method comprises assessing the subject's adherence to antiretroviral therapy (ART). Antiretroviral therapy can be any therapy administered to treat or ameliorate the effects of infection by a virus that incorporates use of reverse transcription in its infection life-cycle. In some embodiments, the ART is administered to treat or ameliorate the effects of infection by a retrovirus. Retroviruses are viruses with RNA genomes that uses a reverse transcriptase to produce a DNA copy of its own genome, which is then typically inserted into the host cell genome. Any retrovirus is contemplated as part of this disclosure. Retroviruses are typically categorized into three groups: oncoretroviruses (oncogenic retroviruses), the lentiviruses (slow retroviruses) and the spumaviruses (foamy viruses). Exemplary, non-limiting human-infecting retroviruses include human immunodeficiency virus (HIV, e.g., HIV-1 and HIV-2, the causative agents of AIDS) and human T-lymphotrophic virus (HTLV). Exemplary, non-limiting veterinary retroviruses include murine leukemia viruses (MLVs), Feline leukemia virus, and Feline immunodeficiency virus, and the like. In such embodiments, a presence of the pharmaceutical agent, or metabolite or derivative thereof in the biological sample as indicated by the method, which is above a pre-set threshold indicates the subject's adherence to ART. Alternatively, a lack of indicated presence of the pharmaceutical agent, or metabolite or derivative thereof, in the biological sample above the pre-set threshold indicates the subject's non-adherence to ART. The pre-set threshold can be established by persons of ordinary skill in the art to reflect amounts of therapeutic agent known to be desirable and/or effective in ART.

As described above, the term “antiretroviral therapy (ART)” can refer to any therapy administered to treat or ameliorate the effects of infection by a virus that incorporates use of reverse transcription in its infection life-cycle. These embodiments cover ART regimens that address infections by viruses that are not retroviruses per se, but do incorporate use of reverse transcription. For example, Hepatitis B virus is not a retrovirus but does use reverse transcription as part of its replication process. Hepatitis B is a partially double-stranded DNA virus that is a species of the genus Orthohepadnavirus and a member of the broader Hepadnaviridae family of viruses. The viral particle has an outer lipid envelope around an icosahedral nucleocapsid proteinaceous core. The nucleocapsid is enclosed around the viral DNA genome and a DNA polymerase that contains reverse transcriptase functionality. During infection and replication, the viral DNA is rendered fully double-stranded by host DNA polymerase and viral mRNAs are transcribed by host RNA polymerase. Additional viral DNA is generated by the viral DNA polymerase from the viral mRNA by virtue of its reverse transcriptase capability. Accordingly, in some embodiments, the ART is administered to treat or ameliorate the effects of infection by a Hepatitis virus by targeting the reverse transcription functionality of the viral DNA polymerase. The Hepatitis virus targeted in the anti-Hepatitis ART can be, for example, Hepatitis A virus, Hepatitis B virus, Hepatitis C virus, and the like. An indicated presence of the pharmaceutical agent, or metabolite or derivative thereof, in the biological sample above a pre-set threshold indicates the subject's adherence to anti-Hepatitis virus therapy. Alternatively, a lack of indicated presence of the pharmaceutical agent, or metabolite or derivative thereof, in the biological sample above the pre-set threshold indicates the subject's non-adherence to anti-Hepatitis virus therapy. The pre-set threshold can be established by persons of ordinary skill in the art to reflect amounts of therapeutic agent known to be desirable and/or effective in the anti-Hepatitis ART. As exemplified by the above Hepatitis B embodiment, the method can be applied to any virus that is not a retrovirus but does depend on reverse transcription for viral replication.

The single stranded nucleic acid template used in the method allows the polymerase in the assay the opportunity to catalyze a complement nucleic acid strand to create a double stranded nucleic acid molecule. The complement nucleic acid strand typically is “primed” by the single stranded nucleic acid primer molecule that hybridizes to the nucleic acid template, where the polymerase serially adds nucleotides from the pool of free dNTPs to the 3′ end of the primer molecule, thus extending the complement strand and creating a lengthening double stranded construct until the polymerase activity is halted. See FIG. 1B.

The single stranded nucleic acid template can be or comprise DNA or can be or comprise RNA. This characteristic of the single stranded nucleic acid template is selected to work in conjunction with the polymerase used in the method. For example, as indicated above various polymerases can be selective for (i.e., produce complement strands from) DNA, whereas others are selective for (i.e., produce complement strands from) RNA. Furthermore, regardless of the primary template, polymerases can also vary with respect to polymerizing DNA or RNA. In some embodiments, the polymerase enzyme has RNA-dependent DNA polymerase properties. In other embodiments, the polymerase enzyme has DNA-dependent DNA polymerase properties. In yet other embodiments, the polymerase enzyme has RNA-dependent RNA polymerase properties.

The single stranded DNA template can be configured for specific activity appropriate for optimal detection of a desired target drug. In some embodiments, the single stranded nucleic acid, e.g., DNA or RNA, template comprises a primer binding domain and a chain terminating domain. The primer binding domain has sufficient complementarity with the single stranded nucleic acid primer molecule such that the single stranded nucleic acid primer molecule will anneal to the primer binding domain of the single stranded nucleic acid under standard hybridization conditions and without substantial off-target hybridization. The chain terminating domain includes bases that are complementary to the target drug or metabolite thereof to increase the likelihood of polymerase inhibition. The chain terminating domain is typically positioned 5′ within the single stranded nucleic acid relative to the primer binding domain. Accordingly, when the single stranded nucleic acid primer molecule anneals to the primer binding domain, the polymerase is able to commence addition of nucleic acid residues to the 3′ end of the primer molecule, thus elongating the complement strand using the sequence of the chain terminating domain as the sequence template. The extent of the elongation process of the complement strand over the chain terminating domain is variable depending on the presence and/or concentration of any inhibitory molecules (e.g., nucleotide analogs) present in the assay. Eventually, a polymerase inhibitor, if present will cause termination of the polymerase activity corresponding to a position within the chain terminating domain of the single stranded nucleic acid template.

The single stranded nucleic acid template can be any length that is minimally sufficient to permit selective binding by the single stranded nucleic acid primer and extension of the complement strand for a sufficient length to provide a dynamic and detectable fluorescent signal. In some embodiments, the single stranded nucleic acid template is at least about 50 nucleotides long. In additional embodiments, the single stranded nucleic acid template is at least about 50, 60, 70, 80, 90, 100, 110, 120, 130, 140, 150, 160, 170, 180, 190, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, 1500, 1600, 1700, 1800, 1900, 2000 or more nucleotides long. The maximum length is not limited except for the practicality and utility of synthesizing the molecules. For example, the single stranded nucleic acid template can have between about at least about 50 total nucleotides and about 2000 (or more) nucleotides. In additional embodiments, the single stranded nucleic acid template has between about 50 and about 1500, between about 50 and about 1250, between about 50 and about 1000, between about 50 and about 750, between about 50 and about 500, between about 50 and about 400, between about 100 and about 1500, between about 100 and about 1250, between about 100 and about 1000, between about 100 and about 750, between about 100 and about 500, between about 100 and about 400, between about 75 and about 300, between about 75 and about 250, between about 75 and about 200, or between about 100 and about 200 nucleotides in length (inclusive of the endpoints), or any other range therebetween.

The single stranded nucleic acid template can be designed, optimized, and/or modified by persons of ordinary skill in the art in a manner to facilitate and/or enhance detection of a selected polymerase inhibitor based on the properties of the polymerase inhibitor and other factors.

To illustrate, in some embodiments the polymerase inhibitor is or comprises a deoxyadenosine triphosphate (dATP) analog, where attempts by the polymerase to incorporate the dATP analog into the growing complement strand effectively terminates the elongation process. Tenofovir diphosphate (TFV-DP) is an illustrative example of such a polymerase inhibitor. The likelihood that the presence of such a polymerase inhibitor is incorporated into the complement strand is influenced by the number of thymine residues in the single stranded nucleic acid template sequence, and more specifically, in the chain terminating domain of the single stranded nucleic acid template. In some examples the thymine content of the single stranded nucleic acid template can be at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%. In some embodiments, the thymine content of the single stranded nucleic acid template can be between about 10% and about 70%, between about 20% and about 60%, between about 25% and about 50%, between about 30% and about 50%, or between about 35% and about 50%. In other embodiments, the thymine content of the single stranded nucleic acid template can be between about 25% and about 80%, between about 25% and about 70%, between about 30% and about 65%, between about 35% and about 60%, or between about 40% and about 60%. The overall thymine content of the single stranded nucleic acid template that can lead to an informative signal is influence by the length of the single stranded nucleic acid template. For example, in longer single stranded nucleic acid templates (e.g., greater than 200 nucleotides in length, such as 400 nucleotides or more in length) the thymine content can be on the lower end of the ranges described above, e.g., with meaningful signal obtained with as little as about 10% thymine residues in the single stranded nucleic acid template. In contrast, shorter single stranded nucleic acid templates (e.g., about 200 nucleotides) require a higher minimal thymine content, such as 20% thymine to provide some meaningful signal.

In other embodiments the polymerase inhibitor is or comprises a dCTP analog, where attempts by the polymerase to incorporate the dCTP analog into the growing complement strand effectively terminates the elongation process. FTC-TP is an illustrative example of such a polymerase inhibitor. The likelihood that the presence of such a polymerase inhibitor is incorporated into the complement strand is influenced by the number of guanine residues in the single stranded nucleic acid template sequence, and more specifically, in the chain terminating domain of the single stranded nucleic acid template. In some examples the guanine content of the single stranded nucleic acid template can be at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%. In some embodiments, the guanine content of the single stranded nucleic acid template can be between about 10% and about 70%, between about 20% and about 60%, between about 25% and about 50%, between about 30% and about 50%, or between about 35% and about 50%. In other embodiments, the thymine content of the single stranded nucleic acid template can be between about 25% and about 80%, between about 25% and about 70%, between about 30% and about 65%, between about 35% and about 60%, or between about 40% and about 60%. As stated above in the context of thymine content parameters, the overall guanine content of the single stranded nucleic acid template that can lead to an informative signal is influenced by the length of the single stranded nucleic acid template. For example, in longer single stranded nucleic acid templates (e.g., greater than 200 nucleotides in length, such as 400 nucleotides or more in length) the guanine content can be on the lower end of the ranges described above, e.g., with meaningful signal obtained with as little as about 10% guanine residues in the single stranded nucleic acid template. In contrast, shorter single stranded nucleic acid templates (e.g., about 200 nucleotides) require a higher minimal guanine content, such as 20% guanine to provide some meaningful signal.

The biological sample can be any biological sample that might contain a polymerase inhibitor, e.g., after administration of the polymerase inhibitor or precursor thereof to a subject. Non-limiting, illustrative examples of biological samples encompassed by this disclosure include blood, serum, plasma, urine, and saliva. Some polymerase inhibitors are known to aggregate within blood cells, e.g., red blood cells or peripheral blood mononuclear cells. Accordingly, in some embodiments, the biological sample comprises red blood cells and/or peripheral blood mononuclear cells. Such samples can include blood or components thereof.

As described in more detail below, whole blood was successfully used as a biological sample to detect polymerase (e.g., reverse transcriptase) inhibitors. Considering the complexity of blood samples and cellular compartmentalization of the inhibitors, the inventors found that the signal detected from the blood could be enhanced by diluting the blood in a matter that lysed the blood cells. Accordingly, in some embodiments, the method further comprises diluting the blood to a final concentration of about 0.1% to about 20% blood. Non-limiting, illustrative dilutions of samples include about 0.1% to about 20% blood, about 0.1% to about 18% blood, about 0.1% to about 15% blood, about 0.1% to about 13% blood, about 0.1% to about 10% blood, about 0.1% to about 9% blood, about 0.1% to about 8% blood, about 0.1% to about 7% blood, about 0.1% to about 6% blood, about 0.1% to about 5% blood, about 0.1% to about 4% blood, about 0.1% to about 3% blood, about 0.1% to about 2% blood, about 0.1% to about 1% blood, about 0.1% to about 0.9% blood, about 0.1% to about 0.9% blood, about 0.1% to about 0.9% blood, about 0.1% to about 0.9% blood, about 0.1% to about 0.8% blood, about 0.1% to about 0.7% blood, about 0.6% to about 0.5% blood, about 0.1% to about 0.4% blood, about 0.1% to about 0.3% blood, or about 0.1% to about 0.2% blood. A person of ordinary skill in the art can readily adjust the other parameters, e.g., concentrations of dNTPs, template, primer, etc., with higher concentrations of blood to be able to obtain useful signals.

In some embodiments, the biological sample is blood and the method further comprises a processing step to denature proteins in the biological sample. For example, the method can further comprise heating the biological sample to above about 70° C., for example above about 75° C., above about 80° C., above about 85° C., above about 90° C., above about 91° C., above about 92° C., above about 93° C., above about 94° C., above about 95° C. for a time sufficient to denature at least some of the protein component in the sample. In some embodiments, the method further comprising removing the denatured protein by, e.g., centrifugation. A person of ordinary skill in the art can readily determine sufficient time for the application of heat. Exemplary times can include at least 3, 4, 5, 6, 7, 8, 9, 10, or more minutes, depending on the temperature. For example, as described in Example 3, the inventors discovered that heating of samples containing a blood component to about 95° C. for up to 10 minutes allowed removal of a substantial amount of denatured protein. The remaining sample resulted in reduced signal variation and, ultimately, a more quantitative measurement of the polymerase inhibitor.

The dNTPs in the assay serve as the building blocks to facilitate elongation of the complement strand by the polymerase to form double stranded molecules that can provide a fluorescent detectable signal. The disclosed method encompasses any functional dNTP concentration that supports such elongation and production of detectable signal. It will be appreciated that the final concentration of the dNTPs can be adjusted based on the concentration of nucleic acid template present to ensure that there is sufficient dNTP to ensure that full-length double stranded DNA can be formed in the absence of inhibitors. The ratio of dNTP to DNA template concentration depends on the sequence of the DNA template and can be readily adjusted by a person of ordinary skill the art. For example, in some embodiments, with a 200 nucleotide long template, the dNTP to template concentration can range from at least 50 to 1 to 200 to 1. Accordingly, in some embodiments, the dNTPs have a final concentration of at least about 20 nM, such as at least about 25 nM, about 30 nM, about 35 nM, about 40 nM, about 45 nM, about 50 nM, about 55 nM, about 60 nM, about 65 nM, about 75 nM, about 80 nM, about 85 nM, about 90 nM, about 95 nM, about 100 nM, about 125 nM, about 150 nM, about 175 nM, about 200 nM, about 225 nM, about 250 nM, about 275 nM, about 300 nM, about 325 nM, about 350 nM, about 375 nM, about 400 nM, about 425 nM, about 450 nM, about 475 nM, about 500 nM, about 550 nM, about 600 nM, about 650 nM, about 700 nM, about 750 nM, about 800 nM, about 850 nM, about 900 nM, about 950 nM, about 1000 nM, or more. In some embodiments, the dNTPs have a final concentration of between about 25 nM to about 1000 nM, such as 25 nM to about 750 nM, 25 nM to about 500 nM, 25 nM to about 250 nM, 50 nM to about 250 nM, 50 nM to about 200 nM, 50 nM to about 150 nM, 75 nM to about 250 nM, 75 nM to about 200 nM, 750 nM to about 150 nM, or any range included therein.

The method encompasses use of any fluorescent dye or moiety that can provide a detectable signal (i.e., fluorescence) with the formation of a double stranded molecule produced by the polymerase using the single stranded nucleic acid as the template. In some embodiments, the fluorescent dye molecule is an intercalating dye molecule. Accordingly, the longer the double stranded molecule is, which reflects a relative paucity of inhibitors, the stronger the detected fluorescent signal is because there is more opportunity for the intercalating dye to integrate into a double stranded nucleic acid molecule. The disclosure encompasses any known intercalating fluorescent dye molecule without limitation. Non-limiting, illustrative examples include PicoGreen, ethidium bromide, SYBR Green, propidium iodide, 7-aminoactinomycin D, EvaGreen, and the like. Persons of ordinary skill in the art can select additional appropriate dyes.

In other embodiments, the fluorescent dye molecule is linked to a probe molecule that can selectively bind the double stranded nucleic acid molecule produced by the polymerase using the single stranded nucleic acid template.

In some embodiments, the probe molecule is an affinity reagent that can selectively bind to the double stranded nucleic acid molecule. As used herein, “affinity reagent” refers to any molecule that can bind a target antigen, in this case the double stranded nucleic acid molecule produced by the polymerase using the single stranded nucleic acid template, with a specific affinity (i.e., detectable over background). Exemplary, non-limiting categories of affinity reagent include antibodies, an antibody-like molecule (including antibody derivatives and fragments (i.e., double stranded DNA-binding fragments thereof)), peptides that specifically interact with a particular antigen (e.g., peptibodies), antigen-binding scaffolds (e.g., DARPins, HEAT repeat proteins, ARM repeat proteins, tetratricopeptide repeat proteins, and other scaffolds based on naturally occurring repeat proteins, etc., [see, e.g., Boersma and Pluckthun, Curr. Opin. Biotechnol. 22:849-857, 2011, and references cited therein, each incorporated herein by reference in its entirety]), aptamers, or a functional double stranded DNA-binding domain or fragment thereof.

In some embodiments, the affinity reagent is an antibody or an antibody-like molecule. As used herein, the term “antibody” encompasses antibodies, derived from any antibody-producing mammal (e.g., mouse, rat, rabbit, and primate including human), that specifically bind to an antigen of interest (e.g., Notch or a cell-type specific antigen). Exemplary antibodies include multispecific antibodies (e.g., bispecific antibodies); humanized antibodies, chimeric antibodies. Antibody-like molecules include any modified antibody, antibody fragment, or molecule comprising an antibody fragment, that retains the functional antigen-binding domain(s) of the antibody. An antibody fragment is a portion derived from or related to a full-length antibody, preferably including the complementarity-determining regions (CDRs), antigen binding regions, or variable regions thereof. Illustrative examples of antibody fragments and derivatives useful in the present disclosure include Fab, Fab′, F(ab)₂, F(ab′)₂ and Fv fragments, nanobodies (e.g., V_{H}H fragments and V_{NAR} fragments), linear antibodies, single-chain antibody molecules, multi-specific antibodies formed from antibody fragments, and the like. Single-chain antibodies include single-chain variable fragments (scFv) and single-chain Fab fragments (scFab). A “single-chain Fv” or “scFv” antibody fragment, for example, comprises the V_{H} and V_{L} domains of an antibody, wherein these domains are present in a single polypeptide chain. The Fv polypeptide can further comprise a polypeptide linker between the V_{H} and V_{L} domains, which enables the scFv to form the desired structure for antigen binding. Single-chain antibodies can also include diabodies, triabodies, and the like.

Production of antibodies or antibody-like molecules can be accomplished using any technique commonly known in the art. Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual (Cold Spring Harbor Laboratory Press, 2nd ed. 1988); Hammerling et al., in: Monoclonal Antibodies and T-Cell Hybridomas 563-681 (Elsevier, N.Y., 1981), incorporated herein by reference in their entireties. The term “monoclonal antibody” refers to an antibody that is derived from a single clone, including any eukaryotic, prokaryotic, or phage clone, and not the method by which it is produced. Methods for producing and screening for specific antibodies using hybridoma technology are routine and well known in the art. Once a monoclonal antibody is identified for inclusion within the bi-specific molecule, the encoding gene for the relevant binding domains can be cloned into an expression vector that also comprises nucleic acids encoding the remaining structure(s) of the bi-specific molecule. Antibody fragments that recognize specific epitopes can be generated by any technique known to those of skill in the art. For example, Fab and F(ab′)₂ fragments of the invention can be produced by proteolytic cleavage of immunoglobulin molecules, using enzymes such as papain (to produce Fab fragments) or pepsin (to produce F(ab′)₂ fragments). F(ab′)₂ fragments contain the variable region, the light chain constant region and the CHI domain of the heavy chain. Further, the antibodies of the present invention can also be generated using various phage display methods known in the art.

In other embodiments, the probe molecule is an affinity reagent selectively binds to a portion of the double stranded nucleic acid molecule. For example, the probe molecule can be a single stranded nucleic acid molecule (DNA, RNA, or a combination thereof), that selectively binds to the complement to the chain terminating domain of the single stranded template molecule. By binding to the complement of this domain, the probe will only bind once a complement strand has been produced by the polymerase, thus providing a signal reflecting the functionality of the polymerase and relative presence or absence of any inhibitors in the assay. In some embodiments, the method further comprises subjecting the biological sample (with the additional reagents) after sufficient time to permit polymerase activity to a denaturing step that facilitates the detectable binding of the fluorescently labeled probe molecule to newly generated complement strands.

Fluorescent dyes and moieties that can be linked to such probe molecules (e.g., affinity reagents and nucleic acid probes, as described above) are known and are encompassed by the present disclosure. Non-limiting, illustrative fluorescent dyes and moieties include hydrolysis probes and molecular beacon probes known in the art. A person of ordinary skill in the art can readily recognize, design, and optimize configurations of the probe-dye combinations to effectuate detection of double stranded nucleic acid molecules that are produced by the polymerase using the single stranded nucleic acid template in the present method.

While the above discussion is presented in the context of exemplary fluorescent dyes, the disclosure also encompasses embodiments that incorporate non-fluorescent dyes as alternatives. For example, detection of polymerase inhibitors via the extent of detectable extension of the complementary strand over the single stranded nucleic acid template molecule can be facilitated by use of nucleic acid probes conjugated to a non-fluorescent small-molecule marker, such as, e.g., biotin, digoxigenin, DNP, and the like. The binding of the probe can be detected or visualized using colorimetric detection in a well or in a lateral flow format. A person of ordinary skill in the art can readily implement such non-fluorescent markers as alternatives to the fluorescent markers in the methods described above.

In another aspect, the disclosure provides a method of assessing the presence of an anti-viral therapeutic agent in a subject receiving pre-exposure prophylaxis (PrEP) or antiretroviral therapy (ART) against a viral infection. The method comprises contacting a biological sample obtained from the subject with the following: a single stranded nucleic acid template, a single stranded nucleic acid primer molecule that hybridizes to the nucleic acid template, reverse transcriptase (RT) enzyme, dNTPs, and a fluorescent dye molecule. The method further comprises providing conditions sufficient to permit the RT enzyme to produce a double stranded nucleic acid molecule by extending a complementary strand along the nucleic acid template, and measuring the fluorescence in the biological sample. A reduced level of measured fluorescence compared to a reference standard indicates the presence of an anti-viral therapeutic agent in the biological sample.

In some embodiments, the method further comprises actively obtaining the biological sample from the subject. The subject can be a human, but can also be another mammal, particularly mammals useful as laboratory models for human disease, e.g., mouse, rat, dog, non-human primate, etc., or animals requiring veterinary treatment or prophylaxis.

PrEP and ART methods are described in more detail above.

The RT enzyme has DNA polymerization activity. In the assay, it is this DNA polymerization activity that is being assessed in the presence of a potential inhibitor, not the reverse transcription function. In an embodiment, the RT enzyme is a nucleotide reverse transcriptase inhibitor, which are described in more detail above. Non-limiting, exemplary nucleotide reverse transcriptase inhibitor metabolites include tenofovir diphosphate (TFV-DP), adefovir diphosphate, and the like. Non-limiting, exemplary nucleoside reverse transcriptase inhibitor metabolites include azidothymidine triphosphate (AZT-TP), emtricitabine triphosphate (FTC-TP), and the like.

The method can further comprise determining a relative concentration or level of the active therapeutic agent in the biological sample. The intensity of fluorescence is inversely correlated to the concentration in active therapeutic agent in the biological sample, as described in more detail above. Therefore, the fluorescent signal can serve as a quantitative or semi-quantitative marker of active therapeutic agent in the biological sample.

The method can further comprise assessing the subject's adherence to pre-exposure prophylaxis (PrEP). Upon performance of the detection and/or quantification, an indicated presence of the anti-viral therapeutic agent in the biological sample above a pre-set threshold indicates the subject's adherence to PrEP. Alternatively, a lack of indicated presence of the anti-viral therapeutic agent in the biological sample above the pre-set threshold indicates the subject's non-adherence to PrEP. For example, the method can comprise assessing the subject's adherence to pre-exposure prophylaxis (PrEP), wherein a level of anti-viral therapeutic agent in the biological sample above a pre-set threshold indicates the subject's adherence to PrEP, and wherein the level of the anti-viral therapeutic agent in the biological sample below the pre-set threshold indicates the subject's non-adherence to PrEP.

Similarly, the method can comprise assessing the subject's adherence to antiretroviral therapy (ART). A detected or inferred level of the anti-viral therapeutic agent in the biological sample above a pre-set threshold indicates the subject's adherence to ART. Alternatively, a detected or inferred level of the anti-viral therapeutic agent in the biological sample below the pre-set threshold indicates the subject's non-adherence to ART. As described in more detail above, antiretroviral therapy can be administered for infections with viruses that incorporate reverse transcription in the replication cycle. In some embodiments, the virus is a retrovirus (e.g., HIV) or is a DNA virus, such as Hepatitis (e.g., Hepatitis B), that uses reverse transcription to replicate its genome in the cell. Thus, the subject can have a retroviral or hepatitis viral infection.

As indicated above, it is the DNA polymerization activity of the RT that is being assessed in the presence of a potential inhibitor and not the reverse transcription function. Therefore, the single stranded nucleic acid template is DNA, RNA, or a combination thereof. The RT enzyme can have RNA-dependent DNA polymerase properties, DNA-dependent DNA polymerase properties, or RNA-dependent RNA polymerase properties, as described in more detail above.

The description provided above to the single stranded DNA template applies to these additional aspects of disclosure. Briefly, in some embodiments, the single stranded DNA template comprises a primer binding domain and a chain terminating domain. When the anti-viral therapeutic agent is or comprises a dATP analog, the chain terminating domain comprises at least 20% thymine residues, for example between about 25% to about 70% thymine residues. When the anti-viral therapeutic agent is or comprises a dCTP analog, the chain terminating domain comprises at least 20% guanine residues, for example between about 25% to about 70% guanine residues. The sensitivity of the assay can be influenced by the total number of thymine (or guanine) residues in the ssDNA template. Thus, for longer ssDNA templates, the minimal percentage of thymine (or guanine) residues can be as low as 20%, where the functional threshold for shorter dsDNA templates may be somewhat higher. This can be readily optimized in view of the various reaction conditions. The single stranded DNA template typically has at least 50 nucleotides, but can be much longer and is limited only by the practicality of synthesizing, handling, and storing longer molecules. In some embodiments, the single stranded DNA template has about 50 nucleotides to about 2000 nucleotides.

As described in more detail above with respect to other aspects, the biological sample is blood, serum, plasma, urine, or saliva. In some embodiments, the biological sample comprises red blood cells and/or peripheral blood mononuclear cells (PBMCs). In embodiments, where the biological sample is or comprises blood, the method can further comprise further manipulation or processing steps. For example, the method can further comprise diluting the blood to a final concentration of about 0.1% to about 20%. Dilution can be made with water or other appropriate liquid buffers. In additional or alternative embodiments, the biological sample that is or comprises a blood component is processed to denature proteins in the sample. For example, the biological sample can be heated at a temperature and for a time sufficient to denature proteins in the biological sample. For example, the biological sample is heated to above about 70° C., about 75° C., about 80° C., about 85° C., about 90° C., or about 95° C. The heating step can last for simply a matter of minutes (e.g., 3, 4, 5, 6, 7, 8, 9, 10 minutes or more). In some embodiments, the higher the applied temperature, the less time is required for the heating step to have its intended effect. In some embodiments, the method further comprises removing the denatured protein by, e.g., centrifugation.

The presence and concentration of dNTPs is described in more detail above and is applicable here. Briefly, the dNTP to nucleic acid template must be sufficient to ensure that full length double stranded DNA can be formed in the absence of inhibitor. In some embodiments, the dNTPs have a final concentration of at least about 20 nM, for example a final concentration of about 25 nM to about 1000 nM, while the nucleic acid templates have a final concentration of at least 0.2 nM, for example a final concentration of about 0.25 nM to about 10 nM.

The fluorescent dye molecule is described in more detail above and is applicable here. Briefly, the fluorescent dye molecule can be an intercalating dye molecule, such as, e.g., PicoGreen, ethidium bromide, SYBR Green, propidium iodide, 7-aminoactinomycin D, EvaGreen and the like. In other embodiments, the fluorescent dye molecule is linked to a probe molecule. Probe molecules and lined fluorescent dyes are described in more detail above and are applicable to this present aspect. While discussion of this aspect is presented in the context of incorporating exemplary fluorescent dyes, the disclosure also encompasses embodiments that incorporate non-fluorescent dyes as alternatives. For example, detection of polymerase inhibitors via the extent of detectable extension of the complementary strand over the single stranded nucleic acid template molecule can be facilitated by use of nucleic acid probes conjugated to a non-fluorescent small-molecule marker, such as, e.g., biotin, digoxigenin, DNP, and the like. The binding of the probe can be detected or visualized using colorimetric detection in a well or in a lateral flow format. A person of ordinary skill in the art can readily implement such non-fluorescent markers as alternatives to the fluorescent markers in the methods described above.

In another aspect, the disclosure provides a kit comprising compositions described herein or compositions to implement methods described herein.

In various embodiments, the disclosed kit can contain a single stranded nucleic acid template, a single stranded nucleic acid primer molecule that hybridizes to the nucleic acid template, a polymerase, dNTPs, and/or a fluorescent dye molecule (or probe with linked fluorescent dye molecule), in any combination. These reagents are described in more detail above. Additional components can include assay buffers and the like. The various reagents are typically contained in sealed vials, bottles, tubes, vials, syringes, or other suitable containers. The reagents can be individually packaged or packaged in batches (e.g., master mixes), as appropriate to retain functionality of the performed method. Individual components can be provided in the kit in concentrated amounts. In some embodiments, a component is provided individually in the same concentration as it would be in a solution with other components. Concentrations of components can be provided as 1×, 2×, 5×, 10×, or 20× or more. The components of the kits can be packaged either in aqueous media or in lyophilized form.

The kit can also contain reference samples with known components of polymerase inhibitor to provide a reference signal or otherwise establish a reference curve of differing known amounts of inhibitor against which experimental results can be compared.

The kit can further include an instructions or directions to electronic forms of instruction on the internet, which outline the procedural steps of the methods set forth herein. The methods typically will follow substantially the same procedures as described herein or are known to those of ordinary skill. The instruction information can be written in tangible form (e.g., on paper) or in a computer readable media containing machine-readable instructions that, when executed using a computer, cause the display of a real or virtual procedure of delivering a pharmaceutically effective amount of a therapeutic agent.

### Additional Definitions

Unless specifically defined herein, all terms used herein have the same meaning as they would to one skilled in the art of the present disclosure. Practitioners are particularly directed to Ausubel, F. M., et al. (eds.), Current Protocols in Molecular Biology, John Wiley & Sons, New York (2010), Coligan, J. E., et al. (eds.), Current Protocols in Immunology, John Wiley & Sons, New York (2010), Mirzaei, H. and Carrasco, M. (eds.), Modern Proteomics—Sample Preparation, Analysis and Practical Applications in Advances in Experimental Medicine and Biology, Springer International Publishing, 2016, and Comai, L, et al., (eds.), Proteomic: Methods and Protocols in Methods in Molecular Biology, Springer International Publishing, 2017, for definitions and terms of art.

For convenience, certain terms employed herein, in the specification, examples and appended claims are provided here. The definitions are provided to aid in describing particular embodiments and are not intended to limit the claimed invention, because the scope of the invention is limited only by the claims.

The use of the term “or” in the claims is used to mean “and/or” unless explicitly indicated to refer to alternatives only or the alternatives are mutually exclusive, although the disclosure supports a definition that refers to only alternatives and “and/or.”

The words “a” and “an,” when used in conjunction with the word “comprising” in the claims or specification, denotes one or more, unless specifically noted.

Unless the context clearly requires otherwise, throughout the description and the claims, the words “comprise,” “comprising,” and the like, are to be construed in an inclusive sense as opposed to an exclusive or exhaustive sense, which is to indicate, in the sense of “including, but not limited to.” Words using the singular or plural number also include the plural and singular number, respectively. The word “about” indicates a number within range of minor variation above or below the stated reference number. For example, “about” can refer to a number within a range of 10%, 9%, 8%, 7%, 6%, 5%, 4%, 3%, 2%, or 1% above or below the indicated reference number.

The term “nucleic acid” refers to a deoxyribonucleotide polymer (DNA) or ribonucleotide polymer (RNA) in either single- or double-stranded form unless specifically defined. The structure of the canonical polymer subunits of DNA, for example, are commonly known and are referred to herein as adenine (A), guanine (G), cytosine (C), and thymine (T). As a group, these are generally referred to herein as nucleotides or nucleotide residues. For RNA, the canonical polymer subunits are the same, except with uracil (U) instead of thymine (T). The nucleic acids can incorporate noncanonical subunits. Illustrative and nonlimiting examples of noncanonical subunits include uracil (for DNA), 5-methylcytosine, 5-hydroxymethylcytosine, 5-formethylcytosine, 5-carboxycytosine b-glucosyl-5-hydroxy-methylcytosine, 8-oxoguanine, 2-amino-adenosine, 2-amino-deoxyadenosine, 2-thiothymidine, pyrrolo-pyrimidine, 2-thiocytidine, or an abasic lesion or site. An abasic site is a location along the deoxyribose backbone that is lacking a base. Known analogs of natural nucleotides hybridize to nucleic acids in a manner similar to naturally occurring nucleotides, such as peptide nucleic acids (PNAs) and phosphorothioate DNA.

Disclosed are materials, compositions, and components that can be used for, can be used in conjunction with, can be used in preparation for, or are products of the disclosed methods and compositions. It is understood that, when combinations, subsets, interactions, groups, etc., of these materials are disclosed, each of various individual and collective combinations is specifically contemplated, even though specific reference to each and every single combination and permutation of these compounds may not be explicitly disclosed. This concept applies to all aspects of this disclosure including, but not limited to, steps in the described methods. Thus, specific elements of any foregoing embodiments can be combined or substituted for elements in other embodiments. For example, if there are a variety of additional steps that can be performed, it is understood that each of these additional steps can be performed with any specific method steps or combination of method steps of the disclosed methods, and that each such combination or subset of combinations is specifically contemplated and should be considered disclosed. Additionally, it is understood that the embodiments described herein can be implemented using any suitable material such as those described elsewhere herein or as known in the art.

Publications cited herein and the subject matter for which they are cited are hereby specifically incorporated by reference in their entireties.

### EXAMPLES

The following examples are set forth so as to provide those of ordinary skill in the art with a complete disclosure and description of how to make and use the present invention, and are not intended to limit the scope of what the inventors regard as their invention nor are they intended to represent that the experiments below are all or the only experiments performed.

### Example 1

### Introduction

This example describes development of a REverSe TRanscrIptase Chain Termination (RESTRICT) assay as a rapid and accessible measurement of drug levels indicative of long term adherence to PrEP and ART.

The assay is inspired by the mechanism of action of TFV-DP on HIV RT and infers drug levels from DNA polymerization. Enzyme inhibition assays targeting RT were originally developed in the context of HIV detection, enzyme characterization, drug screening, and drug resistance monitoring. There are a few reports describing the use of enzyme inhibition assays to measure metabolites of antiretroviral drugs for therapeutic drug monitoring. These early assays all measured incorporation of radio-labeled nucleotides into RNA templates in the presence of antiretroviral drugs extracted from peripheral blood mononuclear cells (PBMCs), which requires labor-intensive and heavily instrumented sample preparation and assay readout that are difficult to implement in routine clinical use. Early enzymatic assays for therapeutic drug monitoring also only targeted metabolites with short half-lives (hours) that were not indicative of long-term adherence.

Building on reports of the accumulation of TFV-DP in red blood cells (RBCs) and pharmacokinetic data about drug levels corresponding to ART and PrEP adherence, the RESTRICT assay was designed to measure antiretroviral drug levels in RBCs with whole blood dilution as a simple sample preparation strategy. The RESTRICT assay was developed and optimized using designer single-stranded DNA templates, primers, and intercalating fluorescence dyes to measure TFV-DP spiked in buffer and blood at clinically relevant concentrations. The RESTRICT assay accurately distinguished TFV-DP concentrations in blood corresponding to low and high long-term PrEP adherence in a simple four-step process in less than 1 hour.

### Experimental Description

### RT Activity Characterization

Optimal assay conditions were determined for RT activity in order to minimize assay time and reagent concentration, using RT enzyme obtained through the NIH AIDS Reagent Program, Division of AIDS, NIAID, NIH (Le Grice, S. F. J.; et al., Purification and Characterization of Human Immunodeficiency Virus Type 1 Reverse Transcriptase. In Methods in Enzymology; DNA Replication; Academic Press, 1995, 262, 130-144). Reactions were carried out in a buffer containing: 60 mM Tris (77-86-1, Sigma), 30 mM KCl (7447-40-7, Sigma), 8 mM MgCl2 (7786-30-3, Sigma), and 10 mM dithiothreitol (20-265, Sigma) buffered to pH 8.0 using HCl (7647-01-0, Acros Organics).

The DNA template has a 20 nt primer binding site complementary to the M13 phage DNA primer AGA GTT TGA TCC TGG CTC AG (Catalog, Integrated DNA Technologies, Coralville, Iowa), set forth herein as SEQ ID NO:1, followed by TTCA repeats with a total template length of 200 nt. The DNA template was designed using NUPACK software (Zadeh, J. N. et al., NUPACK: Analysis and Design of Nucleic Acid Systems. J. Comput. Chem. 2011, 32 (1), 170-173) to preferentially include T bases because TFV-DP is a deoxyadenosine triphosphate (dATP) analog and thus will bind to T's in the DNA template. The template was also designed to be free from secondary structures that could lead to unwanted pausing of the RT enzyme (Huber, H. E. et al., Human Immunodeficiency Virus 1 Reverse Transcriptase. Template Binding, Processivity, Strand Displacement Synthesis, and Template Switching. J. Biol. Chem. 1989, 264 (8), 4669-4678).

To characterize RT activity, master mixes consisting of final concentrations of 5 nM DNA template, 5 nM primer, 50 μM deoxynucleotides (dNTPs) (D7295, Sigma), and RT enzyme concentrations of 25, 50, 100, and 200 nM were prepared in black, flat bottom polystyrene 384-well plates with non-binding surfaces (3575, Corning). RT enzyme was added last after which microwell plates were immediately incubated at 37° C. in a microplate reader (SpectraMax iD3, Molecular Devices). Assays were stopped by manual addition of 40 μL of PicoGreen intercalating dye (P7581, ThermoFisher Scientific) diluted 1:400 in 1×TE (10128-588, VWR). Reactions were quenched at 16-min intervals up to a total time of 128 minutes. PicoGreen was incubated for 1 min before reading out the assay signal with the microplate reader. Assays were run in triplicate unless otherwise specified.

Data was analyzed using GraphPad Prism 8.1 software (GraphPad Software Inc.). The fluorescence intensity from the RT activity assay as a function of time was fit to an exponential curve. Fluorescence intensity as a function of RT enzyme concentration was fit to a four-parameter logistic regression curve that follows the familiar symmetrical sigmoidal shape of enzymatic assays. The four-parameter logistic curve fits take the form:

$$y = D + \frac{A - D}{1 + {( \frac{x}{C} )}^{B}} \qquad ( 1 )$$

For RT activity assays, y represented the fluorescence intensity while x represented the enzyme concentration.

### RESTRICT Assay in Buffer

RESTRICT assays were conducted with TFV-DP (166403-66-3, BOC Sciences Inc.) using 5 μL of DNA template, 5 μL of primer, 20 μL of dNTPs solution, 5 μL of TFV-DP, and 5 μL of HIV-1 RT. Reagent concentrations were varied to optimize experimental conditions (see TABLE 2 in the supplementary information). Serial dilutions of TFV-DP in buffer spanning a concentration range of 1-10,000 nM were prepared to span two orders of magnitude above and below the clinically relevant range for adherence measurement as described in pharmacokinetic studies. See Kearney, B. P. et al., Tenofovir Disoproxil Fumarate. Clin. Pharmacokinet. 2004, 43 (9), 595-612 and Anderson, P. L. et al., Intracellular Tenofovir-Diphosphate and Emtricitabine-Triphosphate in Dried Blood Spots Following Directly Observed Therapy. Antimicrob. Agents Chemother. 2018, 62 (1), e01710-17. RESTRICT assay optimization experiments were completed at 100, 300, 1560, and 6250 nM dNTP concentration.

Fluorescence from RESTRICT assay data was normalized to allow comparison of data points gathered at different dNTP concentrations as follows,

$$\tilde{F} ( j ) = \frac{F ( j ) - {F}_{min}}{{F}_{max} - {F}_{min}} \qquad ( 2 )$$

where the subscripts max and min denote the maximum and minimum measured fluorescence values.

RESTRICT assay data were fit to four-parameter logistic regression curves. The 50% inhibition concentration (IC₅₀)−the concentration of the drug required to achieve 50% inhibition of its target enzyme in vitro was obtained using equation (1) where the parameter x is the TFV-DP concentration and the parameter C represents the IC₅₀.

### RESTRICT Assay in Blood

HIV-negative, human whole blood (BioIVT, Westbury, N.Y.) was diluted in nuclease-free water (3098, Sigma) to lyse RBCs and reduce unwanted inhibition of RT activity by blood components such as immunoglobulins. Blood was mixed with the water by vortexing and incubating for 5 minutes to lyse RBCs.

### Determining Optimal Blood Dilution for RESTRICT

Serial dilutions of blood in water had final blood concentrations ranging from 2% to 10.0%. 5 μL of diluted whole blood at each final concentration was added to 35 μL of master mix (at 500 nM dNTP) to measure RT activity in the presence of diluted blood. Assays were stopped by adding PicoGreen and read out with the plate reader as described previously. Baseline correction was carried out by subtracting the average fluorescence from negative controls (with no RT enzyme) from the fluorescence obtained from each of the RT activity assays.

### RESTRICT Assays in 0.25% Blood

Five μL of TFV-DP were spiked in 2% blood to 35 μL of master mix so that the final concentration of blood in the RESTRICT assay was 0.25%. Serial dilutions of TFV-DP were prepared in diluted blood to correspond with a concentration range of 5.7-11,000 fmol/10⁶ RBCs in whole blood, and thus cover the clinical range for TFV-DP adherence measurement (see TABLE 3 in the supplementary information). Master mixes for the RESTRICT assay in blood contained 2 nM DNA template, 20 nM primer, 100 nM dNTP, and 100 nM of HIV-1 RT. Data corresponding to high and low TFV-DP concentrations within the clinical range for adherence measurement were compared using an unpaired t-test in GraphPad Prism.

### Results and Discussion

The RESTRICT assay measures the average length of cDNA synthesized by RT enzyme in the presence of nucleotide reverse transcriptase inhibitor (NRTI) drugs (FIGS. 1A and 1B). RT forms double-stranded DNA (dsDNA) by polymerizing free nucleotides complementary to a nucleic acid template starting from a region of the template that is hybridized to a primer. At low NRTI concentrations relative to dNTP concentration, RT is unlikely to incorporate NRTIs into the cDNA chain and polymerizes the single-stranded template into full-length dsDNA strands that bind to many intercalating dye molecules and provide a high assay signal. Conversely, at high NRTI concentrations, RT is very likely to incorporate NRTIs into the cDNA chain early, resulting in chain termination and formation of short DNA fragments that bind to fewer intercalating dye molecules and provide a low assay signal. At moderate levels of NRTI, the length of the dsDNA product varies and follows a sigmoidal relationship characteristic of enzyme inhibition assays as shown in FIGS. 1A and 1B. In this way, the fluorescence readout from the RESTRICT assay is used to distinguish low, medium, and high NRTI concentrations.

### RT Activity Characterization

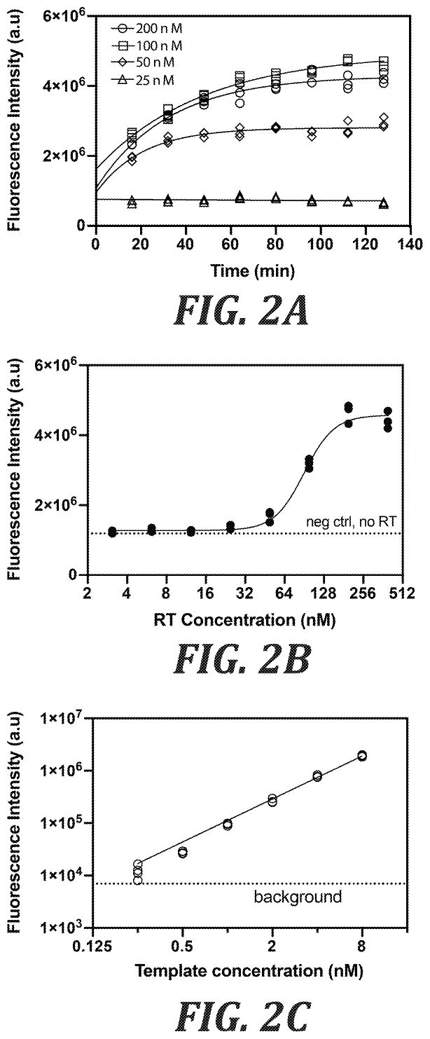

To characterize RT activity, the effect of RT concentration and assay time was determined on the rate of cDNA production as measured by the output fluorescence of an RT activity assay (FIG. 2A). At final concentrations of 50, 100, and 200 nM of RT enzyme, the fluorescence intensity increases with time until ˜60 min, when it plateaus. The fluorescence intensity remains flat over time at 25 nM RT. When optimizing enzyme inhibition assays it is desirable to choose an assay time where RT activity provides measurable fluorescence over baseline levels. The 30 min incubation time provided a strong signal over background levels.

The fluorescence intensity was measured at 8 RT enzyme concentrations to characterize the effect of RT concentration on assay output fluorescence (FIG. 2B). The fluorescence intensity remains at the same level as the negative control (no RT) until ˜25 nM RT when it begins to increase significantly, and then plateaus above ˜200 nM RT. A concentration of 100 nM RT provides an optimal signal over background levels without using excess RT.

The fluorescence output as a function of DNA template concentration was used to determine the lowest template and dNTP concentrations required to measure fluorescence (FIG. 2C). There is a linear relationship between template concentration and fluorescence intensity. The lowest detectable concentration, above background signal, was 0.25 nM of the DNA template.

### RESTRICT Assays in Buffer

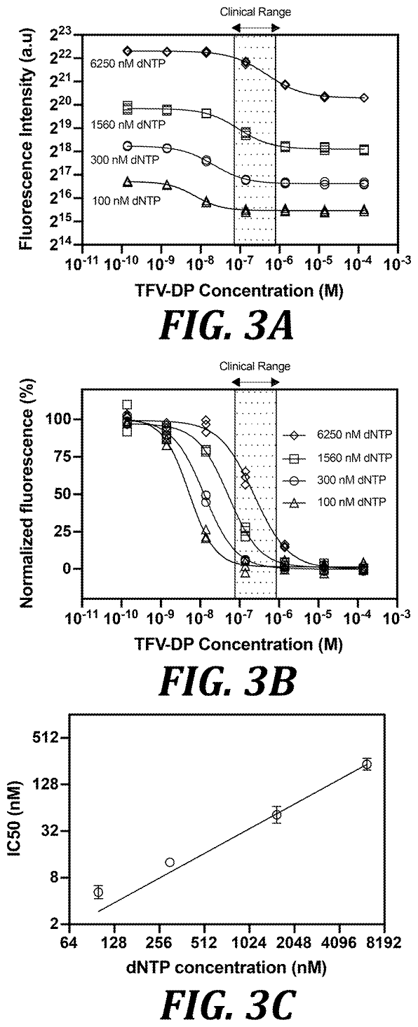

RESTRICT assays were performed with TFV-DP in buffer at concentrations spanning two orders of magnitude above and below the clinical range for PrEP adherence. The RESTRICT assay generates sigmoidal-shaped curves representative of enzyme inhibition assays as a function of TFV-DP concentration (FIG. 3A). As dNTP concentration increases, the fluorescence intensity from the RESTRICT assay increases. This shift in the vertical direction is because a fixed 50 to 1 ratio was kept of dNTP to DNA template in all experiments conducted at 100, 300, 1560, and 6250 nM dNTP. As shown in FIG. 2C, there is a linear relationship between fluorescence intensity and DNA template concentration since more intercalating dyes molecules can be incorporated when there are higher DNA template concentrations.

A shift in the horizontal direction was also observed as dNTP concentration increases. The RESTRICT assay data was normalized to more easily compare inhibition curves at different dNTP concentrations (FIG. 3B). As dNTP concentration increases the inhibition curves shift right, towards higher IC₅₀ values. The IC₅₀ shifts to higher IC₅₀ because dNTP and TFV-DP compete for incorporation into the growing DNA strand and inhibition by lower TFV-DP concentrations can only be detected when there are lower dNTP concentrations.

FIG. 3C shows the measured IC₅₀ values as a function of dNTP concentration and indicates that IC₅₀ values increase linearly as a function of dNTP concentration. This linear relationship allowed the design and optimization of the RESTRICT assay to have a target IC₅₀ value within the clinically relevant concentration range. The RESTRICT assay was designed and optimized to operate at concentration ranges up to two orders of magnitude lower than the clinical range (FIG. 3B), allowing dilution of complex samples (like blood) while still retaining the ability to detect clinically relevant concentrations.

### RESTRICT Assay in Blood

Dilution in water was selected as a simple and user-friendly strategy for sample preparation (Cai, D., et al., Direct DNA and RNA Detection from Large Volumes of Whole Human Blood. Sci. Rep. 2018, 8 (1), 3410) because it both lyses RBCs and also reduces the concentration of confounding blood matrix components that may suppress reverse transcriptase activity. See Cai et al., 2018, supra, and Higuchi, R. Simple and Rapid Preparation of Samples for PCR. In PCR Technology: Principles and Applications for DNA Amplification; Erlich, H. A., Ed.; Palgrave Macmillan UK: London, 1989; pp 31-38.

### Determining Optimal Blood Dilution for RESTRICT

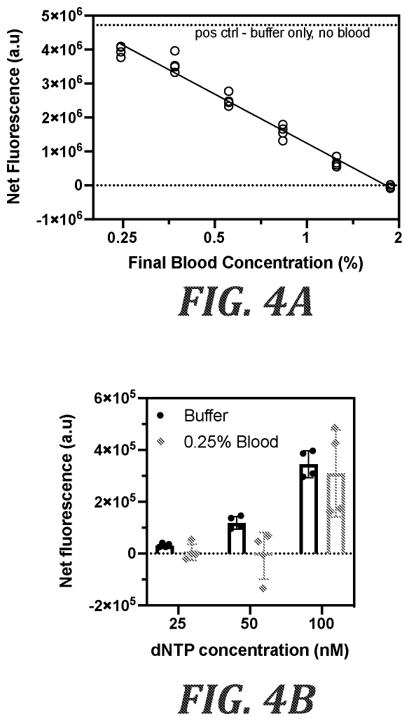

The net fluorescence intensity, i.e. the difference between fluorescence from each RT activity assay and the background signal from no enzyme controls, decreases as blood fraction increases and is indistinguishable from the background at 1.88% final concentration of blood (FIG. 4A). The non-specific inhibition of RT enzyme by blood matrix components also decreases with the concentration of blood in the assay. Thus, diluting blood reduces non-specific inhibition of RT enzyme by blood matrix components (FIG. 4A). However, the trade-off is that greater dilution also decreases the concentration of analyte (TFV-DP) in the sample.

To detect TFV-DP in diluted blood, RESTRICT assays need to be performed at lower IC₅₀ values compared to buffer. The inhibition curve is shifted to lower TFV-DP concentrations by decreasing dNTP concentration (FIGS. 3B and 3C). FIG. 2C shows that the lowest dNTP concentration at which RT activity could be detected in buffer was 25 nM. Anticipating that RT activity in blood would be more variable than in buffer, RT activity assays were conducted in diluted blood to determine the lowest dNTP concentration at which RT activity assays could be performed. A final concentration of 0.25% blood (dilution factor 400×) was selected to minimize non-specific inhibition (FIG. 4A) where there was only a 20% decrease in fluorescence intensity in blood compared with buffer.

The net fluorescence intensity was measured in aqueous buffer and in 0.25% blood at dNTP concentrations of 25, 50, and 100 nM (FIG. 4B). Here the net fluorescence is the difference between the fluorescence measured from each data point minus the signal from a “no RT enzyme” control at the same conditions to account for variations in background signal. FIG. 4B shows that there is a measurable fluorescence signal at 25 nM dNTP in buffer that increases gradually as the dNTP concentration is increased to 50 nM and 100 nM, consistent with FIG. 2C. Conversely, the variation in RT activity when 0.25% blood is introduced means that the net fluorescence is zero at both 25 nM and 50 nM dNTP and RT activity in 0.25% blood is only measurable at 100 nM. Thus, the lowest dNTP concentration that could practically work within 0.25% whole blood was determined to be 100 nM.

### RESTRICT Assays in 0.25% Blood

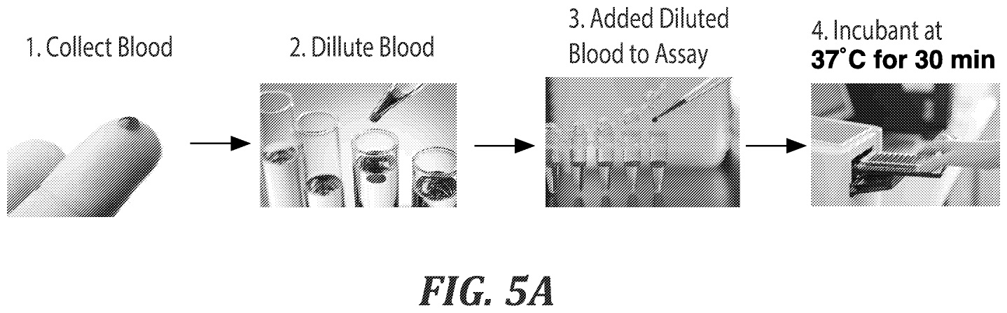

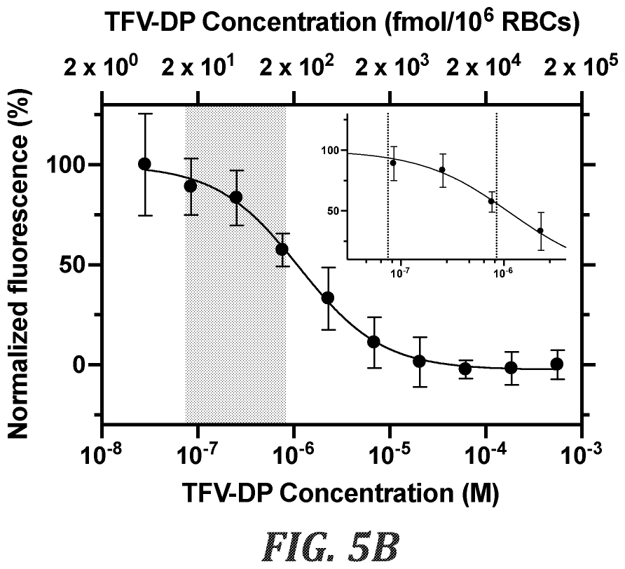

The RESTRICT assay was evaluated for semi-quantitative measurement of clinically relevant TFV-DP concentrations spiked in diluted whole blood. FIG. 5A shows the steps required to complete the RESTRICT assay in blood. The entire assay, from sample collection to assay readout, was completed in less than 1 hour and required <5 μL of blood. FIG. 5B shows data from a RESTRICT assay with 0.25% blood, 100 nM dNTP, at various TFV-DP concentrations around the clinical range for PrEP adherence. The RESTRICT assay data in diluted blood (FIG. 5B) followed the expected sigmoidal shape of enzyme inhibition assays seen in buffer (FIG. 3B); however, there was greater variation in fluorescence intensities in blood compared with buffer. The coefficient of variation of normalized fluorescence from the RESTRICT assay was 11% in blood and only 4% in buffer. This is expected given that blood is a complex sample that contains inhibitors that can suppress reverse transcriptase activity and auto-fluorescent components that can confound intercalating dye signal.

The RESTRICT assay in FIG. 5B overlaps with the clinical range for TFV-DP adherence, although the IC₅₀ of the curve is not located exactly at the center of the clinical range which would maximize the ability to distinguish low and high TFV-DP concentrations within the clinical range. Improved sample preparation to remove unwanted RT inhibition by blood components could allow the use of greater amounts of blood in the assay and enable further optimization of the RESTRICT assay to shift the inhibition curve to the center of the clinical range and reduce the variation when the assay is carried out with blood samples.

Nevertheless, the RESTRICT assay in blood could distinguish drug levels within the clinical range for PrEP adherence measurement. Median TFV-DP concentrations in RBCs range from 15-170 fmol/10⁶ RBCs depending on adherence. See Castillo-Mancilla, J. R., et al., Tenofovir, Emtricitabine, and Tenofovir Diphosphate in Dried Blood Spots for Determining Recent and Cumulative Drug Exposure. AIDS Res. Hum. Retroviruses 2012, 121010062750004. As shown in TABLE 1, the p-value is 0.013 for the unpaired t-test comparing fluorescence at 16.9 fmol/10⁶ RBCs TFV-DP, corresponding to low adherence (1 dose per week), with the fluorescence at 152.3 fmol/10⁶ RBCs TFV-DP, corresponding to high adherence (7 doses per week). These data demonstrate that the RESTRICT assay accurately distinguishes TFV-DP drug levels in blood corresponding to low and high PrEP adherence with high statistical confidence.

TABLE 1
Comparison between RESTRICT assay results
at low and high concentrations within the clinical range
for TFV-DP adherence measurement. N = 4.

| | | |
|---|---|---|
| TFV-DP | 85.4 | 768 |
| Concentration (nM) | | |
| TFV-DP Concentration | 16.9 | 152.3 |
| (fmol/10⁶ RBCs) | | |
| Corresponding Dosage Per | 1 | 7 |
| Week | | |
| Corresponding Adherence | Low | High |
| Level | | |
| Normalized | 89.0 | 57.5 |
| fluorescence (%) | | |
| 95% confidence interval | 66.4-111.5 | 44.1-70.6 |

| | |
|---|---|
| P-value for Unpaired | 0.013 |
| T-Test | |

### Potential Use Cases for the RESTRICT Assay

The RESTRICT assay can provide information on antiretroviral drug levels prior to treatment failure and thus can be a useful and objective tool for monitoring long-term adherence to ART and PrEP in clinical practice and implementation studies. For example, the RESTRICT assay can be used to identify patients with low antiretroviral drug levels (<2 doses per week) (Anderson, P. L., et al., Emtricitabine-Tenofovir Concentrations and Pre-Exposure Prophylaxis Efficacy in Men Who Have Sex with Men. Sci. Transl. Med. 2012, 4 (151), 151ra125-151ra125 and Anderson, P. L., et al., Intracellular Tenofovir-Diphosphate and Emtricitabine-Triphosphate in Dried Blood Spots Following Directly Observed Therapy. Antimicrob. Agents Chemother. 2018, 62 (1), e01710-17) who are at risk of treatment failure. Objective measures of adherence could be used to compare the effectiveness of behavioral interventions designed to improve medication adherence and HIV treatment and prevention outcomes (Castillo-Mancilla, J. R. and Haberer, J. E., Adherence Measurements in HIV: New Advancements in Pharmacologic Methods and Real-Time Monitoring. Curr. HIV/AIDS Rep. 2018, 15 (1), 49-59).

The RESTRICT assay can also be used in conjunction with HIV viral load tests to identify patients at risk of viral rebound or development of drug resistance. Recent work shows that patients with high viral loads and moderately high TFV-DP drug levels are likely to have drug resistant infections. See Yager, J. L., et al., Moderately High Tenofovir Diphosphate in Dried Blood Spots Indicates Drug Resistance in Viremic Persons Living with HIV. J Int. Assoc. Provid. AIDS Care JIAPAC 2019, 18, 1-5. In low- and middle-income countries, where drug resistance tests are inaccessible, HIV positive ART patients who exhibit high viral load levels are often switched to more expensive second or third line drug regimens. See Eholie, S. P., et al., Implementation of an Intensive Adherence Intervention in Patients with Second-Line Antiretroviral Therapy Failure in Four West African Countries with Little Access to Genotypic Resistance Testing: A Prospective Cohort Study. Lancet HIV 2019, 6 (11), e750—e759. The RESTRICT assay can be a useful tool to determine if poor adherence is a contributor to high viral load, and prevent unnecessary use of the second and third line drugs. Furthermore, in settings where viral load measurements are expensive and inaccessible, the RESTRICT assay can be a rapid and inexpensive test that could be performed regularly to determine the risk of treatment failure.

### Conclusion

This investigation demonstrated that the RESTRICT enzymatic assay can be used to measure anti-viral (e.g., ant-HIV) drug levels indicative of long-term PrEP and ART adherence. The assay measures cDNA formation by RT in the presence of TFV-DP. At high TFV-DP concentrations, cDNA chain termination occurs resulting in lower fluorescence signals from intercalating dye. The RESTRICT assay was developed and optimized at TFV-DP concentrations two orders of magnitude above and below the clinical range for PrEP adherence. It is demonstrated that there is a linear relationship between dNTP concentration and the IC₅₀ of the RESTRICT assay and that decreasing dNTP concentration shifts the RESTRICT assay to lower TFV-DP concentrations. TFV-DP was spiked into hemolyzed whole blood at concentrations within the clinical range for adherence measurement and demonstrated that the assay could distinguish concentrations corresponding to low and high PrEP adherence in less than 1 hour. The RESTRICT assay can be a useful test for rapid and accessible measurement of long-term antiretroviral drug levels to identify patients at risk of treatment failure. This work is innovative because it presents a new category of adherence measurement test that can allow patients and clinicians to monitor and improve long-term ART and PrEP adherence and healthcare outcomes.

### Supplemental Information

### Master Mixes for RESTRICT Assays in Buffer

As described in the Experimental Section of the manuscript, master mixes were prepared to optimize RT activity and ensure that the REverSe TRanscrIptase Chain Termination (RESTRICT) assay operates in a regime that provides high assay signal in the absence of nucleotide reverse transcriptase inhibitors (NRTIs). Master mixes for the RESTRICT assay with TFV-DP consisted of DNA template, primer, dNTP solution, TFV-DP, and HIV-1 RT as summarized in TABLE 2 below. Serial dilutions of TFV-DP in buffer spanning a concentration range of 1-10,000 nM were prepared to generate curves representative of TFV-DP inhibition.

TABLE 2
Volumes and concentrations of reagents used
to prepare master mixes for RESTRICT assay.

| | | Volume | | | | | |
|---|---|---|---|---|---|---|---|
| | Stock | per | | Total | Stock | Buffer | Final |
| | Concentration | reaction | # | Vol. | vol. | vol. | Conc. |
| Reagent | [M] | (μL) | Reactions | (μL) | (μL) | (μL) | [M] |
| TCAA | 8.00E−07 | 5 | 29.7 | 148.5 | 3.7 | 144.8 | 2.5E−09 |
| template | | | | | | | |
| 16S | 8.00E−07 | 5 | 29.7 | 148.5 | 37.1 | 111.4 | 2.5E−08 |
| rRNA | | | | | | | |
| primer | | | | | | | |
| dNTP | 5.00E−04 | 20 | 29.7 | 594 | 3.7 | 590.3 | 1.6E−06 |
| | | 30 | 29.7 | 891 | | | |
| TFV-DP | varies | 5 | 29.7 | 148.5 | | | |
| in buffer | | | | | | | |
| HIV-1 | 8.55E−06 | 5 | 26.4 | 132 | 12.4 | 119.6 | 1.0E−07 |
| RT | | | | | | | |
| | | 40 | | 1171.5 | | | |
| After | | | | | | | |
| assay | | | | | | | |
| Picogreen | 400 | 40 | 29.7 | 1188 | 5.9 | 1182.1 | 1 |

### Tenofovir Diphosphate Dilutions in Blood

RESTRICT assays were performed with TFV-DP in whole blood to demonstrate that the RESTRICT assay could operate in a clinically relevant sample and at concentrations relevant for TFV-DP adherence monitoring. Dilution in water was used as a simple sample preparation strategy to lyse red blood cells and reduce non-specific inhibition by blood matrix components. See, e.g., Higuchi, R., Simple and Rapid Preparation of Samples for PCR. In PCR Technology: Principles and Applications for DNA Amplification; Erlich, H. A., Ed.; Palgrave Macmillan UK: London, 1989; pp 31-38 and Cai, D., et al., Direct DNA and RNA Detection from Large Volumes of Whole Human Blood. Sci. Rep. 2018, 8 (1), 3410. To simulate clinical samples, TFV-DP was spiked into the diluted whole blood at concentrations that correspond to the clinical range for TFV-DP adherence. The median concentration of TFV-DP in red blood cells ranges from 15-170 fmol/10⁶ RBCs. See Castillo-Mancilla, J. R., et al., Tenofovir, Emtricitabine, and Tenofovir Diphosphate in Dried Blood Spots for Determining Recent and Cumulative Drug Exposure. AIDS Res. Hum. Retroviruses 2012, 384-390. Assuming an average hematocrit of 40% and an average RBC count of 5×10⁶ RBCs/μL for adults, the clinical range for TFV-DP adherence corresponds to 75-850 nM TFV-DP in whole blood.

To prepare the diluted whole blood at 2% final concentration, 13.3 μL of whole blood was added to 661.7 μL of nuclease-free water. Next, 4.6 μL of 110 μM TFV-DP was added in RT assay buffer to 40.4 μL of the 2% blood to obtain a solution with 11 μM of TFV-DP in 2% blood. The TFV-DP in blood was further diluted by adding 15 μL of 11 TFV-DP in 2% blood to 30 μL of 2% blood. Eight additional 1 in 3 dilutions of TFV-DP in 2% blood were carried as summarized in TABLE 3. TFV-DP concentrations in the assay were chosen so that the corresponding concentration of TFV-DP in undiluted blood (i.e. multiplied by 400 to account for 50× dilution of blood and 8× dilution of TFV-DP in master mix) spanned a range of 5.7-110,000 fmol/10⁶ RBCs and thus covers the clinical range for TFV-DP adherence.

TABLE 3
Serial dilutions of TFV-DP in 2% whole blood.

| | | | | | | | | | | Corresponding |
|---|---|---|---|---|---|---|---|---|---|---|
| | | | | | | | | | Corresponding | conc. in |
| | | | | | | | | | conc. in | undiluted |
| | | | | | | Diluted | Final | Final | undiluted | whole blood |
| | Stock | Vol. per | | Total | Stock | Blood | Conc. | conc. | whole blood | (i.e. 400X) |
| Tube | Conc. | reaction | # | Vol. | vol. | Vol. | in tube | in assay | (i.e. 400X) | [fmol/10⁶ |
| # | (M) | (μL) | Reactions | (μL) | (μL) | (μL) | [M] | [M] | [M] | RBCs] |
| 1 | 1.1E−04 | 5 | 9 | 45 | 4.6 | 40.4 | 1.1E−05 | 1.4E−06 | 5.6E−04 | 1.1E+05 |
| 2 | 1.1E−05 | 5 | 9 | 45 | 15.0 | 30.0 | 3.7E−06 | 4.7E−07 | 1.9E−04 | 3.7E+04 |
| 3 | 3.7E−06 | 5 | 9 | 45 | 15.0 | 30.0 | 1.2E−06 | 1.6E−07 | 6.2E−05 | 1.2E+04 |
| 4 | 1.2E−06 | 5 | 9 | 45 | 15.0 | 30.0 | 4.1E−07 | 5.2E−08 | 2.1E−05 | 4.1E+03 |
| 5 | 4.1E−07 | 5 | 9 | 45 | 15.0 | 30.0 | 1.4E−07 | 1.7E−08 | 6.9E−06 | 1.4E+03 |
| 6 | 1.4E−07 | 5 | 9 | 45 | 15.0 | 30.0 | 4.6E−08 | 5.8E−09 | 2.3E−06 | 4.6E+02 |
| 7 | 4.6E−08 | 5 | 9 | 45 | 15.0 | 30.0 | 1.5E−08 | 1.9E−09 | 7.7E−07 | 1.5E+02 |
| 8 | 1.5E−08 | 5 | 9 | 45 | 15.0 | 30.0 | 5.1E−09 | 6.4E−10 | 2.6E−07 | 5.1E+01 |
| 9 | 5.1E−09 | 5 | 9 | 45 | 15.0 | 30.0 | 1.7E−09 | 2.1E−10 | 8.5E−08 | 1.7E+01 |
| 10 | 1.7E−09 | 5 | 9 | 45 | 15.0 | 30.0 | 5.7E−10 | 7.1E−11 | 2.8E−08 | 5.7E+00 |

### Example 2

This example describes a theoretical model used to support development of the RESTRICT assay described in Example 1.

A theoretical model was developed to measure drug concentration using the principles of the RESTRICT assay (FIGS. 1A and 1B). As described above, the assay requires a nucleic acid template, a primer, free nucleotides (dNTPs), nucleotide reverse transcriptase inhibitors (NRTIs) (e.g. TFV-DP), intercalating dye, and RT enzyme. RT forms double-stranded DNA (dsDNA) by polymerizing a chain of free nucleotides complementary to a nucleic acid template starting from a region of the template that is hybridized to a primer. At low NRTI concentrations relative to dNTP concentration (the top diagram in FIG. 1B), RT is unlikely to incorporate NRTIs into the cDNA chain and can polymerize the ssDNA into full-length dsDNA strands that bind to many intercalating dye molecules and provide a high assay signal. Conversely, at high NRTI concentrations (the bottom diagram in FIG. 1B), RT is very likely to incorporate TFV-DP into the cDNA chain early, resulting in chain termination and formation of short DNA fragments that bind to fewer intercalating dye molecules and provide a low assay signal. At moderate levels of NRTI (the middle diagram in FIG. 1B), the length of the dsDNA product varies and follows a sigmoidal relationship characteristic of enzyme inhibition reactions as shown in FIG. 1B. In this way, the fluorescence readout from the RESTRICT assay is used to distinguish between low, medium, and high NRTI concentrations.

DNA templates were used in the RESTRICT assay because they are less expensive and more stable than RNA templates. It is important to note that although the RESTRICT assay targets the reverse transcriptase (RT) enzyme, the choice to work with DNA rather than RNA templates means that the reverse transcription function of the enzyme is not targeted. Instead, the RESTRICT assay targets the DNA polymerization function of RT enzyme. Nevertheless, the polymerization function of the RT enzyme is also inhibited by NRTIs because of the promiscuity of the RT enzyme in incorporating nucleotide analogs during cDNA formation and its poor error correction capabilities.

### Fluorescence from Full-Length dsDNA Products

As illustrated in FIG. 1B, fluorescence at the end of the RESTRICT assay depends on the interaction between intercalating dye and full-length dsDNA (the top diagram in FIG. 1B), dsDNA fragments (the middle diagram in FIG. 1B), and unpolymerized ssDNA template (the bottom diagram in FIG. 1B). The fluorescence from full-length dsDNA products, F_{fp}, depends on the probability of completion of full-length dsDNA, the length of the DNA template, and the fluorescence properties of the intercalating dye, and can be expressed as,

F_{fp}=Cₜₑₘₚ·K_{dye}·L·P_{dNTP,n} (3)

where P_{dNTp} is the probability that dNTP is inserted into all successive chain termination sites in the cDNA chain, L is the length of a full-length dsDNA product, K_{dye} is a constant that represents the fluorescence per double-stranded base pair per unit concentration provided by the intercalating dye, and Cₜₑₘₚ is the concentration of DNA template in the assay. In this model, L is the number of base-pairs in the entire, full-length dsDNA product, where n corresponds to the total number of bases where NRTI could be inserted (i.e. number of bases complementary to the NRTI in the template strand) and is always less than L. Both n and L depend on the exact sequence of the nucleic acid template used in the RESTRICT assay.

The assay is assumed to be operating at steady state and that dNTP and NRTIs are not depleted during the assay. In addition, the probability that dNTP is inserted into each of the n available NRTI insertion sites in a DNA template is assumed to be an independent event. Thus, the probability of formation of full-length dsDNA, P_{dNTP}, is the probability of a series of n successive dNTP incorporation events which can be calculated using the multiplicative rule for probabilities as,

$${P}_{dNTP , n} = {( \frac{[ dNTP ]}{[ dNTP ] + {K}_{aff} \cdot [ NRTI ]} )}_{1} \cdot {( \frac{[ dNTP ]}{[ dNTP ] + {K}_{aff} \cdot [ NRTI ]} )}_{2} \cdot {( \frac{[ dNTP ]}{[ dNTP ] + {K}_{aff} \cdot [ NRTI ]} )}_{3} … {( \frac{[ dNTP ]}{[ dNTP ] + {K}_{aff} \cdot [ NRTI ]} )}_{n} = {( \frac{[ dNTP ]}{[ dNTP ] + {K}_{aff} \cdot [ NRTI ]} )}^{n} \qquad ( 4 )$$

K_{aff} is the relative affinity of RT for an NRTI compared to its native dNTP substrate, [NRTI] is the concentration of NRTI, and [dNTP] is the concentration of dNTP present in the assay.

Combining equations (3) and (4), the fluorescence from full-length dsDNA can be expressed as,

$${F}_{fp} = {C}_{temp} \cdot {K}_{dye} \cdot L \cdot {( \frac{[ dNTP ]}{[ dNTP ] + {K}_{aff} \cdot [ NRTI ]} )}^{n} \qquad ( 5 )$$

### Fluorescence from Fragment dsDNA Products

Double stranded DNA fragments also contribute to the total fluorescence at the end of the RESTRICT assay (FIG. 1B). Similar to full-length DNA, the fluorescence from dsDNA fragments depends on the probability of formation of a fragment and the length of the DNA fragment. At a given dNTP concentration and NRTI concentration, the probability of chain termination at any of the n possible insertion sites can be calculated for NRTI in the DNA template. Given that a dsDNA fragment is formed whenever there is an NRTI insertion event, the probability of formation of each fragment can be calculated as,

P_{frag,i}=P_{dNTP,i-1}·P_{NRTI} (6)

The index i counts the bases where it is possible to insert NRTI and the maximum value of i is the total number of NRTI insertion bases, or simply n. P_{dNTp,i-1} is the probability that dNTP is incorporated in the nucleic acid template at all bases preceding base i in the template at which NRTI is inserted and can be calculated using equation (5), P_{NRTI} is the probability of NRTI insertion into the nucleic acid template resulting in chain termination and is simply the probability that NRTI is inserted instead of dNTP and expressed as,

$${P}_{NRTI} = 1 - {P}_{dNTP , n = 1} = ( \frac{{K}_{aff} \cdot [ NRTI ]}{[ dNTP ] + {K}_{aff} \cdot [ NRTI ]} ) \qquad ( 7 )$$

Note that P_{dNTP,n=1} and P_{NRTI} are constants for any experimental condition since they represent the probabilities of single insertion events for either dNTP or NRTI, and it is assumed that this assay is working in a regime where reagent depletion is not a concern.

The probability of formation of fragments of sizes ranging from 1 bp to n−1 bp can be calculated, where n is the total number of available NRTI insertion bases in the DNA template. Given the probabilistic nature of dNTP and NRTI insertion, it is expected that there is a distribution of dsDNA fragments sizes at each pair of dNTP and NRTI concentrations. To determine the total fluorescence contribution from dsDNA fragments, the sum of the fluorescence from all the different dsDNA fragment sizes is calculated. Adapting equation (5) and summing the fluorescence from individual dsDNA fragments, the fluorescence from dsDNA fragments can be expressed as follows,

$${F}_{frag} = {C}_{temp} \cdot {K}_{dye} {\sum }_{i = 1}^{n - 1} ( {P}_{dNTP , i - 1} \cdot {P}_{NRTI} \cdot {L}_{i} ) , \qquad ( 8 )$$

where L(i) is the length of the dsDNA fragment when it is terminated at base i and can be deduced from the exact sequence of the DNA template.

### Fluorescence from Unpolymerized ssDNA Template

The fluorescence contribution of intercalating dye interacting with unpolymerized nucleic acid template can be accounted for. At high NRTI concentrations, very little (if any) dsDNA is formed and most of the fluorescence output comes from interactions between the intercalating dye and the unpolymerized ssDNA template (lower diagram of FIG. 1B). Intercalating dyes produce a measurable fluorescence signal when bound to ssDNA fragments. For example, PicoGreen dye used in these experiments, provides 11 times more fluorescence when bound to dsDNA compared to ssDNA. Each dsDNA fragment has a corresponding ssDNA fragment with length equal to the difference between the total length of the nucleic acid template and the dsDNA fragment. Thus, the fluorescence from the ssDNA fragments can be calculated as,

$${F}_{temp} = {C}_{temp} \cdot \frac{{K}_{dye}}{11} \cdot {\sum }_{i = 1}^{n - 1} ( {P}_{NRTI} \cdot {P}_{dNTP , i - 1} \cdot [ L - {L}_{i} ] ) \qquad ( 9 )$$

By combining equations (3)-(9) the total fluorescence of the RESTRICT assay can be calculated as,

Fₜₒₜₐₗ=F_{fp}+F_{frag}+Fₜₑₘₚ (10)

To develop this probabilistic model, it was assumed that the assay operates at steady state and that dNTP and NRTI are not limiting reagents in the reaction. For example, the increase in output fluorescence from full-length product as the template concentration increases that is predicted in equation (4) only holds when sufficient dNTP is available to form full-length dsDNA products with all the available nucleic acid template. The overall goal of the theoretical model was not to obtain exact fluorescence values but rather to understand how shape and position of the inhibition curve (FIG. 1B) changes as assay conditions such as dNTP concentration, TFV-DP concentration, and template concentration change. This allows the probabilistic model to aid in the design of an inhibition assay to quantify TFV-DP within the clinically relevant range of concentrations corresponding to ART and PrEP adherence.

### Example 3

As described in Examples 1 and 2, an objective, near-patient RESTRICT assay was developed that can be used for clinical care. This Example describes an embodiment where the sample preparation procedures are optimized by applying heat to denature proteins in the biological sample.

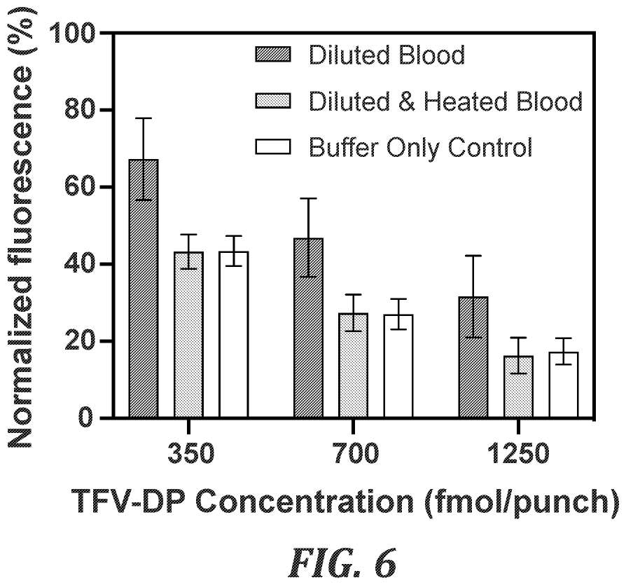

Initial embodiments of the RESTRICT assay can qualitatively distinguish between low and high TFV-DP concentrations. To semi-quantitatively distinguish between low, medium, and high concentrations, alternative sample preparation steps were tested. One approach to achieving semi-quantitative measurements is to reduce the coefficient of variation when working with blood samples. To reduce assay variability, a heating and centrifugation sample preparation step was incorporated. Blood proteins were denatured after dilution by heating to 95° C. for 10 min and centrifuging to separate supernatant (containing TFV-DP) from denatured proteins. Using this improved sample preparation, assay variation was reduced by a factor of two and obtained similar coefficient of variation when the assay was tested with either buffer or blood (FIG. 6). As demonstrated, the RESTRICT assay conducted with sample preparation by diluting and heating blood enables semi-quantitative detection of clinically relevant TFV-DP concentrations.

### Example 4

As described above, maintaining adequate adherence is crucial to obtain the HIV prevention benefits of pre-exposure prophylaxis (PrEP). In this Example, the RESTRICT assay measuring tenofovir diphosphate (TFV-DP) concentrations was compared with liquid chromatography tandem mass spectrometry (LC-MS/MS) as applied to samples obtained from human subjects receiving PrEP for HIV. The results demonstrate that the RESTRICT assay is suitable for measuring HIV RT activity and distinguishing TFV-DP concentrations that correspond to adequate PrEP adherence in clinical samples.

### Introduction

Pre-exposure prophylaxis (PrEP) can prevent HIV acquisition, but maintaining adequate adherence is critical for PrEP efficacy. In several PrEP trials and implementation studies, PrEP clients had difficulties maintaining adequate adherence and persistence and monitoring their PrEP use was challenging. Various indirect and subjective measures have been used to measure adherence, but quantifying concentrations of HIV drugs may provide more objective information for adherence measurements. Tenofovir disoproxil fumarate (TDF), which is used in oral PrEP regimens, is hydrolyzed into tenofovir (TFV) and phosphorylated intracellularly into tenofovir diphosphate (TFV-DP). TFV has a short half-life (17 hours) and is detectable for up to 4 days and 7 days in plasma and urine, respectively. TFV measurement is susceptible to the “white coat” effect where one is unable to distinguish recent pill ingestion from long term adherence. TFV-DP has a longer half-life (17 days), accumulates in red blood cells (RBCs) and peripheral blood mononuclear cells (PBMC), and provides cumulative adherence information over 1-2 months. TFV-DP concentrations have been measured in directly observed therapy (DOT) trials using liquid chromatography tandem mass spectrometry (LC-MS/MS) and established that TFV-DP ≥700 fmol/3 mm punch is commensurate with >4 doses/week on average and provides adequate reduction of HIV incidence risk in the context of PrEP. Although LC-MS/MS provides accurate and quantitative results, it is expensive, laborious, and may be unsuitable for routine clinical use.

As described above, the enzymatic assay, termed REverSe TRanscrIptase Chain Termination (RESTRICT), was developed and optimized for rapid measurement of TFV-DP concentrations. The RESTRICT assay infers drug levels in a patient's blood based on the extent of DNA synthesis by recombinant HIV RT using DNA templates, primers, and nucleotides provided during the assay. The initial results with the RESTRICT assay showed that the assay can accurately distinguish low and high TFV-DP spiked in blood at concentrations corresponding to low and high PrEP adherence. In this study, TFV-DP measurement using the RESTRICT assay was evaluated and compared with TFV-DP measurement by LC-MS/MS in clinical samples to determine whether the RESTRICT assay could distinguish TFV-DP concentrations above the threshold for adequate PrEP adherence.

### Methods

### Study Participants

All study participants were enrolled and sampled in accordance with the University of Washington/Fred Hutch Center for AIDS Research (CFAR) Enhanced Data and Specimen Collection Service. All participants provided informed consent and samples were collected in association with study identifiers. Individuals who receive oral PrEP (TDF+emtricitabine) and individuals not receiving any HIV medication were recruited at the Madison Clinic at Harborview Medical Center in Seattle. Exclusion criteria were age under 18 years, seropositivity for HIV or flavivirus (Zika, Dengue, West Nile, Yellow Fever), or previous enrollment in HIV or flavivirus vaccine study. The following data were collected from each study participant: HIV status, date of birth, sex assigned at birth, query about whether they were taking hormones for gender reassignment, race/ethnicity, body mass index (BMI), and number of doses taken in the past 7 days.

### Blood Sample Collection and LC-MS/MS Measurement

Venous whole blood was collected from each study participant. DBS cards were prepared using 25 μL of each whole blood sample and stored/transported according to previously validated LC-MS/MS protocols (Cressey TR, et al., A randomized clinical pharmacokinetic trial of Tenofovir in blood, plasma and urine in adults with perfect, moderate and low PrEP adherence: the TARGET study. BMC Infectious Diseases. 2017, 17:496). Whole blood tubes were stored on ice prior and analyzed by RESTRICT within 4 hours of sample collection. Matched whole blood and dried blood spot (DBS) samples were tested using the RESTRICT assay and LC-MS/MS, respectively.

### RESTRICT Assay Principle

The RESTRICT assay detects TFV-DP drug concentrations based on its mechanism of action on HIV RT. See examples above and Olanrewaju AO, et al. An enzymatic assay for rapid measurement of antiretroviral drug levels. ACS Sens. April 2020. TFV-DP causes chain termination when HIV RT synthesizes viral double stranded DNA (dsDNA). The RESTRICT assay provides all the reagents required for dsDNA synthesis—a DNA template, primers, nucleotides, recombinant HIV RT enzyme, and appropriate buffers—and measures TFV-DP concentrations in a patient's blood using intercalating fluorescence dyes based on the extent of DNA synthesis. At low TFV-DP concentrations, full-length dsDNA is formed and binds to many intercalating dye molecules resulting in high fluorescence. Meanwhile, at high TFV-DP concentrations, DNA chain termination occurs resulting in dsDNA fragments that bind to few intercalating dye molecules resulting in low fluorescence. Thus, the fluorescence readout from the RESTRICT assay was used to estimate TFV-DP concentration in a patient's blood.

### RESTRICT Assay Reagents and Workflow

Reactions were carried out in a buffer containing: 60 mM Tris (77-86-1, Sigma), 30 mM KCl (7447-40-7, Sigma), 8 mM MgCl₂ (7786-30-3, Sigma), 10 mM dithiothreitol (20-265, Sigma), 400 nM deoxynucleotide triphosphates (dNTPs) (D7295, Sigma), 40 nM primer 16S rRNA Forward primer AGA GTT TGA TCC TGG CTC AG (51-01-19-06, Integrated DNA Technologies, Coralville, Iowa), set forth herein as SEQ ID NO:1, and 4 nM DNA template buffered to pH 8.0 using HCl (7647-01-0, Acros Organics). Custom-designed DNA templates were synthesized in silico (Integrated DNA Technologies, Coralville Iowa) and consisted of a 20 nucleotide (nt) primer binding site followed by a 180 nt detection region with TTCA repeats. Recombinant reverse transcriptase protein obtained through the NIH AIDS Reagent Program, Division of AIDS, NIAID, NIH (Le Grice SFJ, et al., Purification and characterization of human immunodeficiency virus type 1 reverse transcriptase. In: Methods in Enzymology. Vol 262. DNA Replication. Academic Press; 1995:130-144).

The RESTRICT assay was completed in 5 simple steps. First the collected blood was diluted to 8% volume in nuclease-free water (3098, Sigma-Aldrich), mixed and vortexed for 5 min to lyse red blood cells (RBCs), release intracellular TFV-DP, and reduce assay inhibition by blood components. Then 5 μL of diluted whole blood was added to 30 μL of buffered master mix in flat-bottom polystyrene 384-well plates with nonbinding surfaces (3575, Corning). Then 5 μL of 100 nM of HIV-1 RT was added to initiate the assay which was incubated at 37° C. for 30 min in a microplate reader (SpectraMax iD3, Molecular Devices). PicoGreen™ dye (P7581, ThermoFisher Scientific) diluted 1:400 in 1×TE (10128-588, VWR) was added to stop the reaction and provide fluorescence output. 5 replicates were tested for each sample.

Buffer only controls were run with each set of experiments. A standard curve was generated by running the RESTRICT assay with 5 aliquots of TFV-DP spiked into diluted blood (from participant 001, not on PrEP) at final concentrations corresponding to 9 to 58333 fmol/punch and spanning nearly two orders of magnitude above and below the PrEP adherence clinical range.

### Statistical Analysis

The standard curve was used to determine the limit of detection (LoD) of the RESTRICT assay in blood using the equation: LoD=μ_{B}−1.645σ_{B}−1.645σₛ, where μ_{B} is the mean intensity of the negative control samples, σ_{B} is the intensity standard deviation of the negative control samples, and σₛ is the intensity standard deviation of the low concentration spiked samples. This LoD formula was adapted from recommendations by the Clinical and Laboratory Standards Institute (CLSI) (Tholen D W. Protocols for Determination of Limits of Detection and Limits of Quantitation: Approved Guideline. Wayne, Pa.: NCCLS; 2004 and Borysiak M D, et al., Translating diagnostic assays from the laboratory to the clinic: analytical and clinical metrics for device development and evaluation. Lab Chip. 2016;16(8):1293-1313) since negative controls (with no TFV-DP) provide high (100%) signal while high TFV-DP concentrations provide low (0%) signal in the RESTRICT assay.

Baseline correction was carried out by subtracting baseline fluorescence from each sample (with no RT enzyme) from endpoint assay fluorescence (after RT incubation for 30 min) to account for variations in baseline fluorescence of each sample. The fluorescence intensity from each sample was normalized by dividing by the fluorescence obtained with the buffer control. A receiver operating characteristic (ROC) curve was prepared to identify samples with LC-MS/MS TFV-DP concentration ≥700 fmol/punch. The Spearman correlation coefficient between RESTRICT fluorescence and LC-MS/MS TFV-DP concentrations was also calculated.

### Results

In total, 18 individuals were included [4 (22%) women, median age 56 years; interquartile range (IQR) 48 to 56] (TABLE 4). All 11 participants not receiving PrEP had undetectable (<200 fmol/punch) TFV-DP by LC/MS (TABLE 4). Six out of 7 participants receiving PrEP had detectable TFV-DP levels and four out of seven of the same participants had TFV-DP levels ≥700 fmol/punch.

TABLE 4
Demographic characteristics and LC-MS/MS
measurements of study participants.

| | PrEP | No PrEP |
|---|---|---|
| | (N = 7) | (N = 11) |
| Median age (IQR) | 50 (45 to 62) | 57 (52 to 65) |
| Body mass index, BMI (kg/m²) | 25 (23 to 27) | 31 (23 to 37) |
| Number of women (%) | 1 (14%) | 3 (27%) |
| LC-MS TFV-DP | 717, 2248, 2453, | All |
| concentration | 2556, 675, 559, | undetectable |
| (fmol/punch) | undetectable | |
| Median RESTRICT | 39.5% | 51.3% |
| fluorescence intensity | (27.9 to 50.3%) | (49.9 to 62.8%) |
| (95% CI) | | |

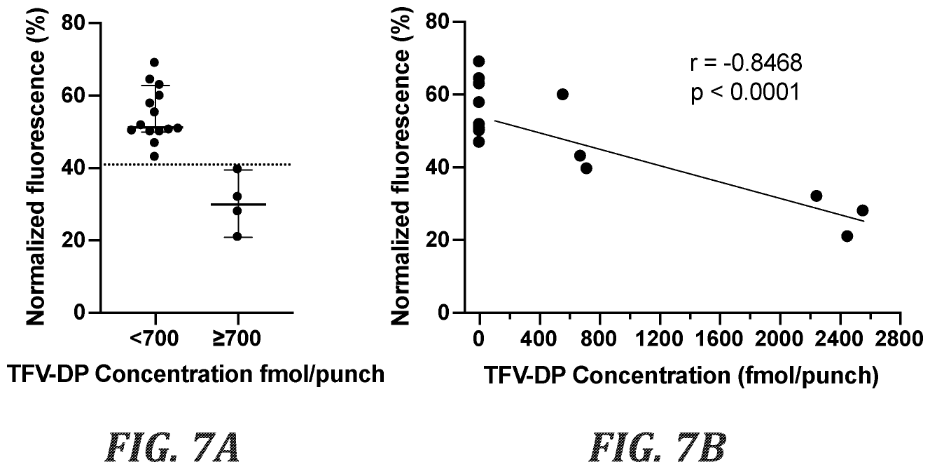

The limit of detection of the RESTRICT assay in diluted blood was 201.4 fmol/punch (95% CI: 108.1 to 334.8). Overall, the median fluorescence from the RESTRICT assay was higher for participants receiving PrEP compared to participants not receiving PrEP (TABLE 4). Median fluorescence was 51.3% (95% CI: 49.9 to 62.8%) for samples containing <700 fmol/punch (N=14) and 31.9% (95% CI: 20.8 to 39.5%) for samples containing ≥700 fmol/punch (N=4). Applying a fluorescence threshold of 41.2% yielded 100% specificity and 100% sensitivity in identifying participants with TFV-DP concentrations ≥700 fmol/punch (FIG. 7A). RESTRICT assay fluorescence intensities were correlated with LC-MS/MS TFV-DP concentrations, r=−0.8468 (95% CI: −0.9456 to −0.6051), p<0.0001 (FIG. 7B).

### Discussion

Here, that RESTRICT assay fluorescence is demonstrated to be is correlated with LC-MS/MS TFV-DP measurements in a cohort of adults receiving PrEP at the Madison Clinic in Seattle. Fluorescence levels were significantly lower in individuals with TFV-DP concentrations above the threshold for adequate PrEP adherence (≥700 fmol/punch) compared with individuals with lower or undetectable TFV-DP concentrations.

Measuring antiretroviral concentrations provides accurate long-term adherence information that is correlated with clinical outcomes. Urine TFV tests have been developed by the inventors and others for rapid adherence measurement, but they only measure recent medication ingestion and can be subject to white-coat effect. TFV-DP concentrations in RBCs indicate long term adherence and can be measured using LC-MS/MS; however, LC-MS/MS is complex, time-consuming, and expensive. The RESTRICT represents a new class of rapid, objective, long-term adherence test that can be completed using reagents and equipment that are available in most clinical laboratories.

The present results, in view of the results from the Examples above incorporating spiked blood samples provide preliminary evidence for the potential of the RESTRICT assay for rapid detection of antiretroviral concentrations in clinical settings.

The RESTRICT assay can be used to evaluate the role of adherence in treatment failure and emergence of drug resistance among people living with HIV. The RESTRICT assay can also be useful to screen eligible HIV vaccine trial candidates who have been taking PrEP in order to increase efficiency. Beyond adherence, the RESTRICT assay can be used for drug monitoring in clinical trials to study the relationship between drug concentration, efficacy, and toxicity for individual trial participants.

### Conclusion

The RESTRICT assay, a rapid, objective test for TFV-DP concentrations that correlate with long-term PrEP adherence, was evaluated for practical application. The RESTRICT assay identified participants with TFV-DP concentrations above the threshold for adequate adherence with good agreement with gold standard LC-MS/MS measurement. The RESTRICT assay has critical utility to fill the gap of rapid long-term adherence measurement to promote more honest conversations about PrEP use and enable improved PrEP counselling.

While illustrative embodiments have been illustrated and described, it will be appreciated that various changes can be made therein without departing from the spirit and scope of the invention.

1. A method of detecting a polymerase inhibitor in a biological sample, comprising:
contacting the biological sample with
a single stranded nucleic acid template,
a single stranded nucleic acid primer molecule that hybridizes to the nucleic acid template,
a polymerase,
dNTPs, and
a fluorescent dye molecule;
providing conditions sufficient to permit the polymerase to produce a double stranded nucleic acid molecule by extending a complementary strand along the nucleic acid template; and
measuring fluorescence in the biological sample;
wherein a reduced level of measured fluorescence compared to a reference standard indicates the presence of a polymerase inhibitor in the biological sample.

2. The method of claim 1, wherein the polymerase inhibitor is a pharmaceutical agent, or metabolite or derivative thereof, and the biological sample is obtained from a subject.

3. The method of claim 2, wherein the polymerase inhibitor is a reverse transcriptase inhibitor or a metabolite thereof.

4. The method of claim 3, wherein the reverse transcriptase inhibitor is a nucleotide reverse transcriptase inhibitor, a nucleoside reverse transcriptase inhibitor, or a metabolite thereof.

5. (canceled)

6. The method of claim 3, wherein the metabolite of the reverse transcriptase inhibitor is tenofovir diphosphate (TFV-DP), azidothymidine triphosphate (AZT-TP), emtricitabine triphosphate (FTC-TP), lamivudine triphosphate (3TC-TP), adefovir diphosphate, or entecavir triphosphate.

7. The method of claim 1, further comprising determining a relative concentration of polymerase inhibitor in the biological sample, wherein intensity of fluorescence is inversely correlated to the concentration of polymerase inhibitor in the biological sample.

8. The method of claim 2, wherein the method comprises assessing the subject's adherence to pre-exposure prophylaxis (PrEP), wherein an indicated presence of the pharmaceutical agent, or metabolite or derivative thereof, in the biological sample above a pre-set threshold indicates the subject's adherence to PrEP, or wherein a lack of indicated presence of the pharmaceutical agent, or metabolite or derivative thereof, in the biological sample above the pre-set threshold indicates the subject's non-adherence to PrEP.

9. The method of claim 2, wherein the method comprises assessing the subject's adherence to antiretroviral therapy (ART), wherein an indicated presence of the pharmaceutical agent, or metabolite or derivative thereof, in the biological sample above a pre-set threshold indicates the subject's adherence to ART, or wherein a lack of indicated presence of the pharmaceutical agent, or metabolite or derivative thereof, in the biological sample above the pre-set threshold indicates the subject's non-adherence to ART.

10. The method of claim 2, wherein the method comprises assessing the subject's adherence to anti-Hepatitis virus therapy, wherein an indicated presence of the pharmaceutical agent, or metabolite or derivative thereof, in the biological sample above a pre-set threshold indicates the subject's adherence to anti-Hepatitis virus therapy, or wherein a lack of indicated presence of the pharmaceutical agent, or metabolite or derivative thereof, in the biological sample above the pre-set threshold indicates the subject's non-adherence to anti-Hepatitis virus therapy.

11-15. (canceled)

16. The method of claim 1, wherein the single stranded nucleic acid template is DNA, wherein the single stranded DNA template comprises a primer binding domain and a chain terminating domain.

17. The method of claim 16, wherein the polymerase inhibitor is or comprises a dATP analog and wherein the chain terminating domain comprises at least 20% thymine residues, or wherein the polymerase inhibitor is or comprises a dCTP analog and wherein the chain terminating domain comprises at least 20% guanine residues.

18-20. (canceled)

21. The method of claim 16, wherein the single stranded DNA template has at least 50 nucleotides.

22. (canceled)

23. The method of claim 1, wherein the biological sample is blood, serum, plasma, urine, or saliva.

24. (canceled)

25. The method of claim 23, wherein the biological sample is blood and the method further comprises diluting the blood to a final concentration of about 0.1% to about 20%.

26. The method of claim 23, wherein the biological sample is blood and the method further comprises heating the biological sample to above about 70° C.

27. The method of claim 1, wherein the dNTPs have a final concentration of at least about 20 nM.

28. (canceled)

29. The method of claim 1, wherein the fluorescent dye molecule is an intercalating dye molecule.

30. (canceled)

31. The method of claim 1, wherein the fluorescent dye molecule is linked to a nucleic acid probe.

32. (canceled)

33. A method of assessing the presence of an anti-viral therapeutic agent in a subject receiving pre-exposure prophylaxis (PrEP) or antiretroviral therapy (ART) against a viral infection, comprising:
contacting a biological sample obtained from the subject with
a single stranded nucleic acid template,
a single stranded nucleic acid primer molecule that hybridizes to the nucleic acid template,
a reverse transcriptase (RT) enzyme,
dNTPs, and
a fluorescent dye molecule;
providing conditions sufficient to permit the RT enzyme to produce a double stranded nucleic acid molecule by extending a complementary strand along the nucleic acid template; and
measuring the fluorescence in the biological sample;
wherein a reduced level of measured fluorescence compared to a reference standard indicates the presence of an anti-viral therapeutic agent in the biological sample.

34. (canceled)

35. The method of claim 33, wherein the anti-viral therapeutic agent is a nucleotide reverse transcriptase inhibitor agent or metabolite thereof and is selected from tenofovir diphosphate (TFV-DP) and adefovir diphosphate, or wherein the anti-viral therapeutic agent is a nucleoside reverse transcriptase inhibitor or metabolite thereof and is selected from azidothymidine triphosphate (AZT-TP), lamividuine triphosphate (3TC-TP), and emtricitabine triphosphate (FTC-TP).

36-64. (canceled)
